(19) United States
(12) Patent Application Publication
(10) Pub. No.: US 2026/0226155 A1
(43) Pub. Date: Aug. 6, 2026

(54) Anti-ADM antibodies, anti-ADM antibody fragment or anti-ADM non-Ig scaffold for therapy or prevention of immune effector cell therapy side effects

(71) Applicant: PAM Theragnostics GmbH, Hennigsdorf (DE)

(72) Inventors: Yulia Ilina, Berlin (DE); Paul Kaufmann, Berlin (DE)

(73) Assignee: PAM Theragnostics GmbH, Hennigsdorf (DE)

(21) Appl. No.: 19/310,790

(22) Filed: Aug. 26, 2025

Related U.S. Application Data
(60) Provisional application No. 63/687,230, filed on Aug. 26, 2024.
(60) Provisional application No. 63/687,239, filed on Aug. 26, 2024.

(30) Foreign Application Priority Data
Aug. 26, 2024 (EP) 24196442.8
Aug. 26, 2024 (EP) 24196494.9

(51) Int. Cl. C07K 16/26; A61K 39/00; A61K 45/06

(52) U.S. Cl. CPC C07K 16/26; A61K 45/06; A61K 2039/505; C07K 2317/565; C07K 2317/76; C07K 2317/92

(57) ABSTRACT

The present application is directed to an anti-ADM antibody or anti-ADM antibody fragment or anti-ADM non-Ig scaffold for use in therapy or prevention of side effects of immune effector cell administration to a cancer patient in the frame of an immune effector cell therapy (IECT), wherein the anti-ADM antibody or anti-ADM antibody fragment or anti-ADM non-Ig scaffold is administered prior to or after administration of the immune effector cells to the patient.

### PRIORITY CLAIM

This application claims priority to U.S. Provisional Patent Application No. 63/687,230 filed on Aug. 26, 2024 and titled “ANTI-ADM ANTIBODIES, ANTI-ADM ANTIBODY FRAGMENT OR ANTI-ADM NON-IG SCAFFOLD FOR THERAPY OR PREVENTION OF IMMUNE EFFECTOR CELL THERAPY SIDE EFFECTS,” U.S. Provisional Patent Application No. 63/687,239 filed on Aug. 26, 2024 and titled “A METHOD FOR THE PREDICTION OF A RISK OR SEVERITY OF SIDE EFFECTS IN A CANCER PATIENT THAT IS TO BE TREATED OR HAS BEEN TREATED WITH IMMUNE EFFECTOR CELL THERAPY (IECT),” European Patent Application No. EP24196442.8 filed on Aug. 26, 2024 and titled “ANTI-ADM ANTIBODIES, ANTI-ADM ANTIBODY FRAGMENT OR ANTI-ADM NON-IG SCAFFOLD FOR THERAPY OR PREVENTION OF IMMUNE EFFECTOR CELL THERAPY SIDE EFFECTS,” and European Patent Application No. EP24196494.9 filed on Aug. 26, 2024 and titled “A METHOD FOR THE PREDICTION OF A RISK OR SEVERITY OF SIDE EFFECTS IN A CANCER PATIENT THAT IS TO BE TREATED OR HAS BEEN TREATED WITH IMMUNE EFFECTOR CELL THERAPY (IECT),” the entire contents of all of which are hereby incorporated by reference herein.

### SEQUENCE LISTING

The instant application contains a Sequence Listing which has been submitted electronically in XML format and is hereby incorporated by reference in its entirety. SaidXML copy, created on Aug. 26, 2025, is named P75635EP.xml and is 37,952 bytes in size.

### FIELD OF THE INVENTION

The invention relates to an anti-ADM antibody or anti-ADM antibody fragment or anti-ADM non-Ig scaffold for use in therapy or prevention of side effects of immune effector cell administration to a cancer patient in the frame of an immune effector cell therapy (IECT), wherein said anti-ADM antibody or anti-ADM antibody fragment or anti-ADM non-Ig scaffold is administered prior to or after administration of said immune effector cells to said patient.

### BACKGROUND

### (Pro-)Adrenomedullin

Adrenomedullin (ADM) is one of the best studied peptide hormones, which plays a role in a vast range of physiological and pathophysiological processes, including inter alia vasodilation, angiogenesis, and hormone regulation. ADM is also involved in bronchodilatation, renal function, cell growth, differentiation, neurotransmission, and modulation of the immune response. ADM belonging to the ADM/calcitonin gene-related peptide (CGRP) superfamily of peptides and is known to be produced in various human organs and tissues, including the heart, adrenal endothelial cells, lungs, kidneys, adipose tissue, and vascular endothelium, which contribute to ADM blood levels.

ADM mRNA encodes a preprohormone of 185 amino acids (SEQ ID No.: 1), the pre-pro-Adrenomedullin that is enzymatically converted into Proadrenomedullin by cleavage of the N-terminal signal peptide. Proadrenomedullin (SEQ ID No.: 2) is then further process by several prohormone convertases to result in four peptides, namely
PAMP-Gly (SEQ ID No.: 3): Proadrenomedullin N-terminal 20 peptide with a C-terminal glycine residue, inactive precursor of biologically active PAMP (PAMP-NH₂ (SEQ ID No.: 4)),
MR-proADM (SEQ ID No.: 5): Mid-regional Proadrenomedullin, a stable and inert peptide,
ADM-Gly (SEQ ID No.: 6): C-terminally glycine extended, inactive precursor of biologically active ADM (mature ADM/ADM-NH₂ (SEQ ID No.: 7) and
CT-proADM (SEQ ID No.: 8): C-terminal Proadrenomedullin or Adrenotensin.

ADM-Gly is the direct, inactive biosynthetic precursor of the fully activated ADM form (mature ADM), often referred to as the intermediate form of ADM, and represents the dominating circulating form of ADM in humans. To gain its biological activity, ADM-Gly is activated by the Vitamin C dependent enzyme peptidylglycine-alpha amidating monooxygenase (PAM). PAM recognizes the C-terminal glycine and catalyses a sequential two-step reaction also referred to as amidation or C-terminal amidation.

Mature ADM has several physiological effects, such as vasodilation, angiogenesis, cardioprotection, nephroprotection, anti-oxidation, anti-apoptosis and tissue repair and regeneration. Mature ADM is involved in blood pressure regulation, bronchodilatation, renal function, hormone secretion, cell growth, differentiation, neurotransmission, and modulation of the immune response. Moreover, ADM plays a crucial role as autocrine factor during proliferation and regeneration of endothelial cells.

Additionally, mature ADM promotes angiogenesis, arteriogenesis, prevents cognitive decline after chronic cerebral hypoperfusion and is therefore considered as therapeutic agent in vascular dementia (reviewed in (Garcia et al., 2006) and (Bálint et al., 2023).

ADM is an effective vasodilator, and thus it is possible to associate the hypotensive effect with the particular peptide segments in the C-terminal part of ADM. It has furthermore been found that the above-mentioned physiologically active peptide PAMP formed from pre-proADM likewise exhibits a hypotensive effect, even if it appears to have an action mechanism differing from that of ADM (in addition to the above-mentioned review articles (Eto, 2001) and Hinson et al. 2000 see also Kuwasaki et al. 1997. FEBS Lett 414(1): 105-110; Kuwasaki et al. 1999. Ann. Clin. Biochem. 36: 622-628; Tsuruda et al. 2001 Life Sci. 69(2): 239-245 and EP-A2 0 622 458). It has furthermore been found that the concentrations of ADM, which can be measured in the circulation and other biological liquids, are in a number of pathological states, significantly above the concentrations found in healthy control subjects. Thus, the ADM level in patients with congestive heart failure, myocardial infarction, kidney diseases, hypertensive disorders, diabetes mellitus, in the acute phase of shock and in sepsis and septic shock are significantly increased, although to different extents. The PAMP concentrations are also increased in some of said pathological states, but the plasma levels are lower relative to ADM Eto 2001. Peptides 22: 1693-1711). It was reported that unusually high concentrations of ADM are observed in sepsis, and the highest concentrations in septic shock (Eto 2001. Peptides 22: 1693-1711, Hirata et al. Journal of Clinical Endocrinology and Metabolism 81(4): 1449-1453; Ehlenz et al. 1997. Exp Clin Endocrinol Diabetes 105: 156-162; Tomoda et al. 2001. Peptides 22: 1783-1794; Ueda et al. 1999. Am. J. Respir. Crit. Care Med. 160: 132-136 and Wang et al. 2001. Peptides 22: 1835-1840).

WO2004/097423 describes the use of an antibody against adrenomedullin for diagnosis, prognosis, and treatment of cardiovascular disorders. Treatment of diseases by blocking the ADM receptor are also described in the art, (e.g., WO2006/027147, PCT/EP2005/012844) said diseases may be sepsis, septic shock, cardiovascular diseases, infections, dermatological diseases, endocrinological diseases, metabolic diseases, gastroenterological diseases, cancer, inflammation, hematological diseases, respiratory diseases, muscle skeleton diseases, neurological diseases, urological diseases.

### Endothelial Dysfunction

Endothelial (barrier) dysfunction has been described for a number of diseases and is considered to contribute to the pathogenesis of these diseases. These include pulmonary diseases, ARDS occurring in COVID-19 and in asthma, arthritis, ulcerative colitis, other chronic inflammatory diseases, cancer, age-related macular degeneration, diabetic macular edema, psychiatric and neurodegenerative diseases, cardiovascular diseases, circulatory shock including septic shock, hepatic and renal diseases, infectious and autoimmune diseases, (Claesson-Welsh et al., 2021)(Rodrigues & Granger, 2015)(O Karpinich et al., 2011)(Soussi et al., 2023)(Opal & Van Der Poll, 2015)(W. L. Lee & Slutsky, 2010)(Chistiakov et al., 2015)(Claesson-Welsh, 2015).

### Endothelial Dysfunction and Adrenomedullin

Elevation of plasma ADM is widely accepted as a surrogate marker for disturbed endothelial barrier function. High ADM levels likely represent a failing compensatory response, aimed at restoring endothelial barrier function (van Lier et al., 2020). Elevated plasma concentrations have been described in most of the diseases, which are associated with endothelial dysfunction (see above).

### Immune Effector Cell Therapy

Over the past decades, an increasing number of ways to treat cancer has been developed (Link 2019). These include chemotherapy, hormone therapy, hyperthermia, photodynamic therapy, radiation therapy, stem cell transplant, surgery, targeted therapy, and immunotherapy. Recently, Immune Effector Cell Therapies (IECT) have emerged as the most promising therapy in oncology (Forero-Forero et al. 2021). Under the term IECT several types of cell therapies are summarized, which include Chimeric Antigen Receptor T-cell (CAR T) therapy, natural killer cell (NK) therapy, chimeric antigen receptor natural killer cell (CAR-NK) therapy, T cell receptor-engineered T cell (TCR T) therapy, tumor-infiltrating T cell (TIT)), and cytokine-induced killer cell (CIK) therapy, of which CAR T-cell therapy has been studied and used the most (Forero-Forero et al. 2021). CAR T-cell therapy has turned out to be particularly effective in the treatment of patients with B cell malignancies (Sun et al. 2024). Currently, CAR T-cell therapy is approved for the treatment of B cell relapsed or refractory leukemia and lymphoma, and most recently, multiple myeloma. In these different diseases, it has led to excellent complete and overall response rates depending on the patient population and therapy. The field of CAR T-cell therapy continues to expand rapidly. IECTs other than CAR T, including CAR-NK, TIL, CIK are being explored in hematologic malignancies and solid malignancies such as germ cell tumor, sarcoma, neuro-blastoma, and melanoma (Kanate et al. 2023).

Despite promising efficacy, CAR T cell therapy as well as the other IECTs mentioned above are frequently associated with significant side effects (Lee et al. 2019). The two most notable toxicities are cytokine release syndrome (CRS) and immune effector cell-associated neurotoxicity syndrome (ICANS) (Chohan, Siegler, and Kenderian 2023).

CRS has been defined as “a supraphysiologic response following any immune therapy that results in the activation or engagement of endogenous or infused T cells and/or other immune effector cells. Symptoms can be progressive, must include fever at the onset, and may include hypotension, capillary leak (hypoxia) and end organ dysfunction. (Lee et al. 2019)”.

ICANS has been defined as “a disorder characterized by a pathologic process involving the central nervous system following any immune therapy that results in the activation or engagement of endogenous or infused T cells and/or other immune effector cells. Symptoms or signs can be progressive and may include aphasia, altered level of consciousness, impairment of cognitive skills, motor weakness, seizures, and cerebral edema (Lee et al. 2019)”. Similar to CRS, ICANS should be applied to any immune effector cell engaging therapy, not just CAR T-cells.

CRS is one of the most common side effects associated with CAR T-cell therapy with an incidence of 20-50% and manifests as fever and difficulty of breathing, low blood pressure, nausea and vomiting, and poses a notable safety challenge and can lead to life-threatening conditions, such as hypotension, respiratory distress and organ insufficiency (Sun et al. 2024).

Regimes have been developed to treat CAR T-cell-induced toxicity. These include supportive care, the use of tocilizumab, an IL-6 receptor antibody, and corticosteroids, if patients are refractory to tocilizumab. Even with the use of tocilizumab, which is FDA approved to treat severe CRS, severe CRS and death still occur.

For the treatment of neurotoxicity, tocilizumab is not effective, and steroids are used as first line of treatment there. The pathophysiology and mechanisms underlying neurotoxicity are only barely understood (Neelapu et al. 2018).

Other agents which have been investigated and are generally reserved for refractory CRS include anti-IL-6 antibody (siltuximab), anti-TNF antibody (etanercept, infliximab) and anti-IL-1 antibody (anakinra). They all aim at downregulating the overactivation of the immune system.

Due to the side-effects of IECTs and the so far limited possibilities to treat these, experts have called for novel toxicity-directed therapies and low-toxicity constructs which do not compromise treatment efficacy, which are desperately needed (Chohan, Siegler, and Kenderian 2023).

The immune system emerges as a key player not only mediating cytokine responses but potentially contributing to endothelial injury in CAR T-cell toxicity (Gavriilaki et al. 2020). Thus, endothelial dysfunction has been investigated in CAR T-cell toxicity (Sumransub et al. 2022). Following CAR T-cell therapy, endothelial dysfunction characterized by increased pro-infalmmatory signaling (e.g., IL-6, IL-8, MCP-1) loss of barrier function has been observed, and CAR T-cells have been shown to directly induce endothelial dysfunction (Rosen et al. 2023).

The administration of CAR T-cells is preceded by a chemotherapy to achieve lymphodepletion, which is applied typically for three days, and subsequently, after a rest of two days the CAR T-cells are infused.

One study has been published, in which the influence of chemotherapy on the levels of Adrenomedullin was investigated (Güler et al. 2006). The study was restricted to pediatric patients and did not comprise a following CAR T-cell therapy. The authors reported slightly higher levels of Adrenomedullin after chemotherapy compared to baseline (30.3 pmol/mL vs 25.1 pmol/mL).

At this point, there are no publications describing biomarker status prior to immune effector cell administration (in particular CAR T-cell administration) in the frame of an immune effector cell threrapy (in particular in the frame of a CAR T-cell therapy) or shortly after administration of immune effector cells (in particular CAR T-cells) in the frame of an immune effector cell therapy (in particular in the frame of a CAR T-cell therapy), which would predict an increased risk for endothelial dysfunction, which would develop as a side effect of immune effctor cell therapy (in particular CAR T-cell therapy), nor how to prevent such side-effect.

After side effects, i.e. CRS, have occurred and treatment of side effects has been initiated, there is then during the course of treatment uncertainty about when CRS is considered resolved (Lee et al. 2019).

Under the state of the art, once such therapies are administered, the patient is considered to still have CRS, even in the absence of fever, until all signs and symptoms leading to the diagnosis of CRS are considered resolved, which puts additional burden, e.g. on medical personnel and on the patient due to excessive need for patient surveillance. Thus, there is the need for better tools to monitor the success of treatment of side effects resulting from IECTs.

WO2013/072510 describes a non-neutralizing anti-ADM antibody for use in therapy of a severe chronical or acute disease or acute condition of a patient for the reduction of the mortality risk for said patient.

WO2013/072511 describes a non-neutralizing anti-ADM antibody for use in therapy of a chronical or acute disease or acute condition of a patient for prevention or reduction of organ dysfunction or organ failure.

WO2013/072512 describes a non-neutralizing anti-ADM antibody that is an ADM stabilizing antibody that enhances the half-life (t1/2 half retention time) of adrenomedullin in serum, blood, plasma. This ADM stabilizing antibody blocks the bioactivity of ADM to less than 80%.

WO2013/072513 describes a non-neutralizing anti-ADM antibody for use in therapy of an acute disease or condition of a patient for stabilizing the circulation.

WO2013/072514 describes a non-neutralizing anti-ADM antibody for regulating the fluid balance in a patient having a chronic or acute disease or acute condition.

### DESCRIPTION OF THE INVENTION

It is the surprising finding of the present invention that increased proADM concentrations (e.g. ADM-Gly or bio-ADM concentrations) prior to immune effector cell administration in the frame of an immune effector cell therapy, which is indicative of endothelial dysfunction, means that patients are more likely to develop side effects of the immune effector cell therapy, whereas lower proADM concentrations are indicative of patients not developing such side effects, or that increased proADM concentrations (e.g. ADM-Gly or bio-ADM concentrations) prior to immune effector cell administration in the frame of a immune effector cell therapy means that patients have a higher risk to develop side effects of the immune effector cell therapy, whereas lower proADM concentrations means that patients have a lower risk to develop such side effects.

Consequently, such patients presenting with endothelial dysfunction prior to immune effector cell administration in the frame of an immune effector cell therapy would benefit from a therapy improving the endothelial function prior to immune effector cell administration. Such treatment of endothelial dysfunction would be for example the treatment with anti-ADM antibodies binding to the N-terminal proportion of ADM, such as enibarcimab. Therefore, it is plausible that cancer patients presenting with endothelial dysfunction and thereby having an increased risk to develop side effects due to the immune effector cell therapy represent a target group for the treatment with anti-ADM antibodies, such as Enibarcimab.

Subject of the the present invention is thus an anti-ADM antibody or anti-ADM antibody fragment or anti-ADM non-Ig scaffold for use in therapy or prevention of side effects of immune effector cell administration to a cancer patient in the frame of an immune effector cell therapy (IECT), wherein said anti-ADM antibody or anti-ADM antibody fragment or anti-ADM non-Ig scaffold is administered prior to or after administration of said immune effector cells to said patient.

Further subject matter of the present invention is an anti-ADM antibody or anti-ADM antibody fragment or anti-ADM non-Ig scaffold for use in in therapy or prevention of side effects prior to or after immune effector cell administration to a cancer patient for in the frame of a immune effector cell therapy, wherein said side effects of administration of said immune effector cells to said patient are associated with endothelial dysfunction.

In embodiments of the invention the therapy or prevention of side effects as detailed herein in a cancer patient prior to immune effector cell administration in the frame of immune effector cell therapy is therapy or prevention of side effects as detailed herein in a cancer patient scheduled to receive (i.e. to be administered) immune effector cell therapy, and the anti-ADM antibody or anti-ADM antibody fragment or anti-ADM non-Ig scaffold is to be administered prior to immune effector cell administration, i.e. wherein said patient has not yet received (i.e. been administered) immune effector cells.

In embodiments of the present invention said anti-ADM antibody or anti-ADM antibody fragment or anti-ADM non-Ig scaffold is used for reducing immune effector cell therapy side effects.

In embodiments of the present invention, “prior to immune effector cell administration” or “prior to immune effector cell administration in the frame of a immune effector cell therapy” means in particular that the anti-ADM antibody or anti-ADM antibody fragment or anti-ADM non-Ig scaffold is to be administered in the time span between lymphodepletion and administration of the immune effector cells, more particularly within of 14 days, or within 7 days, or within 2 days before said immune effector cells are administered to said patient, even more particularly within of 48 hours, or within 24 hours, of within 12 hours, or within 6, or within 2 hours, or within 1 hour before said immune effector cells are administered to said patient; in other words, said patient is scheduled for administration of immune effector cells within the aforementioned time frames after administration of the anti-ADM antibody or anti-ADM antibody fragment or anti-ADM non-Ig scaffold.

In embodiments of the present invention, “after administration of immune effector cells” or “after administration of said immune effector cells in the frame of an immune effector cell therapy” means in particular that the anti-ADM antibody or anti-ADM antibody fragment or anti-ADM non-Ig scaffold is to be administered to said patient that has received said treatment, before said patient has developed side effects from said treatment or alternatively after said patient has developed side effects from said treatment.

In a specific embodiment said anti-ADM antibody or anti-ADM antibody fragment or anti-ADM non-Ig scaffold is to be administered to said patient that has received said treatment and has developed side effects from said treatment within 24 hours, or within 12 hours, or within 6, or within 3 hours, or within 2 hours, or within one hour or within 30 minutes or within 15 min after onset of said side effects.

In particular embodiments of the present invention said patient is having a level of Pro-Adrenomedullin or fragments thereof in a sample of bodily fluid above a certain threshold.

In more particular embodiments of the present invention said patient is having a level of Pro-Adrenomedullin or fragments thereof in a sample of bodily fluid above a certain threshold and wherein said Pro-Adrenomedullin or fragments thereof is selected from the group comprising Pro-Adrenomedullin (SEQ ID No.: 2), PAMP-Gly (SEQ ID No.: 3), mature PAMP (SEQ ID No.: 4), MR-proADM (SEQ ID No.: 5), ADM-Gly (SEQ ID No.: 6), mature ADM (SEQ ID No.: 7) and CT-proADM (SEQ ID No.: 8).

In particular embodiments of the present invention said Pro-Adrenomedullin or fragment thereof is selected from the group comprising Pro-Adrenomedullin (SEQ ID No.: 2), mature PAMP (SEQ ID No.: 4), PAMP-Gly (SEQ ID No.: 3), MR-proADM (SEQ ID No.: 5), ADM-Gly (SEQ ID No.: 6), mature ADM (SEQ ID No.: 7) and CT-proADM (SEQ ID No.: 8).

Mature ADM, bio-ADM and ADM-NH₂ is used synonymously throughout this application and is a molecule according to SEQ ID No.: 7.

In more particular embodiments of the present invention said patient is having a level of Pro-Adrenomedullin or fragments thereof in a sample of bodily fluid above a certain threshold and wherein said fragment of Pro-Adrenomedullin is mature ADM (SEQ ID No.: 7) and the threshold of the level of mature ADM in a sample of bodily fluid of said patient is between 35 and 125 μg/ml, more preferred between 40 and 100 μg/ml, even more preferred between 50 and 90 μg/ml, most preferred said threshold is 70 μg/ml.

In more particular embodiments of the present invention said patient is having a level of Pro-Adrenomedullin or fragments thereof in a sample of bodily fluid above a certain threshold and wherein said fragment of Pro-Adrenomedullin is ADM-Gly (SEQ ID No.: 6) and the threshold of the level of ADM-Gly in a sample of bodily fluid of said patient is between 25 and 125 μg/ml, more preferred between 30 and 100 μg/ml, even more preferred between 35 and 75 μg/ml, most preferred said threshold is 40 μg/ml.

In more particular embodiments of the present invention said patient is having a level of Pro-Adrenomedullin or fragments thereof in a sample of bodily fluid above a certain threshold and wherein said fragment of Pro-Adrenomedullin is MR-proADM (SEQ ID No.: 5) and the threshold of the level of MR-proADM in a sample of bodily fluid of said patient is between 0.5 and 2 nmol/L, more preferred between 0.6 and 1.5 nmol/L, even more preferred between 0.7 and 1 nmol/L, most preferred said threshold is 0.8 nmol/L.

In more particular embodiments of the present invention said patient is having a level of Pro-Adrenomedullin or fragments thereof in a sample of bodily fluid above a certain threshold and wherein said fragment of Pro-Adrenomedullin is mature PAMP (SEQ ID No.: 4) and the threshold of the level of mature PAMP in a sample of bodily fluid of said patient is between 0.7 and 1.2 pmol/L, more preferred between 0.8 and 1.0 pmol/L, most preferred said threshold is 0.9 pmol/L.

In more particular embodiments of the present invention said patient is having a level of Pro-Adrenomedullin or fragments thereof in a sample of bodily fluid above a certain threshold and wherein said fragment of Pro-Adrenomedullin is PAMP-Gly (SEQ ID No.: 3) and the threshold of the level of PAMP-Gly in a sample of bodily fluid of said patient is between 0.5 and 2.5 pmol/L, more preferred between 0.7 and 2.0 pmol/L, even more preferred between 1.0 and 1.8 pmol/L, most preferred said threshold is 1.5 pmol/L.

In more particular embodiments of the present invention said fragment of Pro-Adrenomedullin is MR-proADM and the threshold of the level of MR-proADM in a sample of bodily fluid of said patient is between 0.5 and 2 nmol/L, more particular between 0.6 and 1.5 nmol/L, even more particular between 0.7 and 1 nmol/L, most particular said threshold is 0.8 nmol/L, or is an x-fold of the median level of MR-proADM in a healthy population, in particular in the range between 1.2-fold and 4.9-fold, more particular between 1.5-fold and 3.7-fold, even more particular between 1.7-fold and 2.4-fold, most particular said threshold is 2.0-fold of the median of the level of MR-proADM in a healthy population.

In more particular embodiments of the present invention said fragment of Pro-Adrenomedullin is mature PAMP and the threshold of the level of mature PAMP in a sample of bodily fluid of said patient is between 0.7 and 1.2 pmol/L, more particular between 0.8 and 1.0 pmol/L, most particular said threshold is 0.9 pmol/L, or is an x-fold of the mean level of mature PAMP in a healthy population, in particular in the range between the 1.4-fold and 2.4-fold, more particular in the range between 1.6-fold and 2.0-fold, most particular said threshold is the 1.8-fold of the mean of the level of mature PAMP in a healthy population.

In more particular embodiments of the present invention said fragment of Pro-Adrenomedullin is PAMP-Gly and the threshold of the level of PAMP-Gly in a sample of bodily fluid of said patient is between 1.5 and 2.4 pmol/L, even more preferred between 1.8 and 2.2 pmol/L, most preferred said threshold is 2 pmol/L, or is an x-fold of the mean level of PAMP-Gly in a healthy population, in particular in the range between the 1.3-fold and 2.1-fold, more particular in the range between 1.6-fold and 1.9-fold, most particular said threshold is the 1.7-fold of the mean of the level of PAMP-Gly in a healthy population.

In more particular embodiments of the present invention said fragment of Pro-Adrenomedullin is ADM-Gly and the threshold of the level of ADM-Gly in a sample of bodily fluid of said patient is between 25 and 125 μg/ml, more preferred between 30 and 100 μg/ml, even more particular between 35 and 75 μg/ml, most particular said threshold is 40 μg/ml, or is an x-fold of the median level of ADM-Gly in a healthy population, in particular in the range between the 0.9-fold and 4.6-fold, more particular in the range between the 1.1-fold and 3.7-fold, more particular in the range between 1.3-fold and 2.8-fold, most particular said threshold is the 1.5-fold of the median of the level of ADM-Gly in a healthy population.

In more particular embodiments of the present invention said fragment of Pro-Adrenomedullin is mature ADM and the threshold of the level of mature ADM in a sample of bodily fluid of said patient is between and 125 μg/ml, more particular between 40 and 100 μg/ml, even more particular between 50 and 90 μg/ml, most particular said threshold is 70 μg/ml, or between the 2.6-fold and 9.1-fold, more particular in the range between the 2.9-fold and 7.3-fold, more particular in the range between 3.6-fold and 6.6-fold, most particular said threshold is the 5.1-fold of the median of the level of mature ADM in a healthy population.

In more particular embodiments of the present invention said fragment of Pro-Adrenomedullin is CT-proADM and the threshold of the level of CT-proADM in a sample of bodily fluid of said patient is between 75 and 350 pmol/L, more particular between 100 and 250 pmol/L, even more particular between 125 and 200 pmol/L, most particular said threshold is 150 pmol/L, or is an x-fold of the median level of CT-proADM in a healthy population, in particular in the range between the 1.0-fold and 4.5-fold, more particular in the range between the 1.3-fold and 3.2-fold, more particular in the range between 1.6-fold and 2.6-fold, most particular said threshold is the 1.9-fold of the median of the level of CT-proADM in a healthy population.

In more particular embodiments of the present invention said patient is having a level of Pro-Adrenomedullin or fragments thereof in a sample of bodily fluid above a certain threshold and wherein the level of proADM or fragments thereof is determined by contacting said sample of bodily fluid with a capture binder that binds specifically to proADM or fragments thereof.

In more particular embodiments of the present invention said sample is a bodily fluid sample selected from the group comprising whole blood, plasma or serum.

In even more particular embodiments of the present invention said patient is having a level of Pro-Adrenomedullin or fragments thereof in a sample of bodily fluid above a certain threshold and wherein the sample of bodily fluid of said patient is selected from the group of whole blood, serum and plasma.

In more particular embodiments of the present invention said patient is having a level of Pro-Adrenomedullin or fragments thereof in a sample of bodily fluid above a certain threshold and is determined using a point-of-care device.

In more particular embodiments of the present invention said patient is having a level of Pro-Adrenomedullin or fragments thereof in a sample of bodily fluid above a certain threshold and wherein said point-of-care device is a microfluidic device.

As used herein, a microfluidic device has a plurality of chambers arranged at different positions which are connected in parallel and into which a fixed amount of fluid may be efficiently distributed without using a separate driving source, wherein said device includes a platform having a center of rotation and including at least one microfluidic structure. Microfluidic devices are used to perform biological or chemical reactions by manipulating small amounts of fluid.

In a preferred embodiment, the bio-ADM level is determined from a plasma sample of said patient. It is however typical in the technical lifecycle improvement of measurement of analytes that possibilities exist to measure such analytes in other—in particular blood-based—matrices. For instance, in case of bio-ADM, another technology has been developed, which uses whole (EDTA-) blood, known as IB10 Sphingotest® bio-ADM (https://www.nexus-dx.com/wp-content/uploads/2020/07/bio-ADM-IFU-REV-A.pdf). The IB10 Sphingotest® bio-ADM® is a rapid point-of-care (POC) immunoassay for the in vitro quantitative determination of human amidated adrenomedullin peptide (1-52), in the following referred to as bioactive adrenomedullin (bio-ADM®), in human EDTA whole blood and plasma.

A bodily fluid according to the present invention is in one particular embodiment a blood sample. A blood sample may be selected from the group comprising whole blood, serum and plasma. In a specific embodiment of the invention said sample is selected from the group comprising human citrate plasma, heparin plasma and EDTA plasma.

In one embodiment the analytical assay sensitivity of said assay for ADM-Gly is able to quantify ADM-Gly of healthy subjects and is 20 μg/ml, preferably 15 μg/ml and more preferably 10 μg/ml.

In one embodiment the analytical assay sensitivity of said assay for ADM-Gly is able to quantify ADM-Gly of healthy subjects and is a 0.7-fold, preferably a 0.6-fold and more preferably 0.4-fold of the median of a healthy population.

In one embodiment the analytical assay sensitivity of said assay for PAMP is able to quantify PAMP of healthy subjects and is <0.5 pmol/L, preferably <0.25 pmol/L and more preferably <0.1 pmol/L.

In one embodiment the analytical assay sensitivity of said assay for PAMP is able to quantify PAMP of healthy subjects and is a 1.0-fold, preferably a 0.5-fold and more preferably a 0.2-fold of the mean of a healthy population.

In one embodiment the analytical assay sensitivity of said assay for the detection of CT-proADM is able to quantify CT-proADM of healthy subjects and is <100 pmol/L, preferably <75 pmol/L and more preferably <50 pmol/L.

In one embodiment the analytical assay sensitivity of said assay for the detection of CT-proADM is able to quantify CT-proADM of healthy subjects and is a 1.3-fold, preferably a 1.0-fold and more preferably a 0.6-fold of the median of a healthy population.

In one embodiment the analytical assay sensitivity of said assay for the detection of mature ADM is able to quantify mature of healthy subjects and is <40 μg/ml, preferably <25 μg/ml and more preferably <10 μg/ml.

In one embodiment the analytical assay sensitivity of said assay for the detection of mature ADM is able to quantify mature of healthy subjects and is a 2.9-fold, preferably a 1.8-fold and more preferably a 0.7-fold of the median of a healthy population.

In one embodiment the analytical assay sensitivity of said assay is able to quantify MR-proADM of healthy subjects and is <0.5 nmol/L, preferably <0.4 nmol/L and more preferably <0.2 nmol/L.

In one embodiment the analytical assay sensitivity of said assay is able to quantify MR-proADM of healthy subjects and is a 1.2-fold, preferably a 1.0-fold and more preferably a 0.5-fold of the median of a healthy population.

In a specific embodiment, said binder exhibits a binding affinity to proADM or fragments thereof of at least 10⁷ M⁻¹, preferred 10⁸ M⁻¹, preferred affinity is greater than 109 M⁻¹, most preferred greater than 10¹⁰ M⁻¹. A person skilled in the art knows that it may be considered to compensate lower affinity by applying a higher dose of compounds and this measure would not lead out-of-the-scope of the invention.

To determine the affinity of the antibodies to proADM or fragments thereof, the kinetics of binding of Adrenomedullin to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare), (Lorenz et al. 2011. Antimicrob Agents Chemother. 55 (1): 165-173).

In a specific embodiment, said binder is selected from the group comprising an antibody or an antibody fragment or a non-Ig scaffold binding to proADM or fragments thereof.

In a specific embodiment an assay is used for determining the level of proADM or fragments thereof, wherein such assay is a sandwich assay, preferably a fully automated assay.

In one embodiment such assay for determining the concentration (mass per volume) of proADM or fragments thereof is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems: Roche Elecsys®, Abbott Architect®, Siemens Advia Centauer®, Siemens Immulite®, Brahms Kryptor®, Biomerieux Vidas®, Alere Triage®, Boditech AFIAS®, Ortho Vidas®, Diasorin LIASION®, Beckman Dxl®, Lumira Dx®, MeMed Key®, Werfen BioFlash®, BioRad BioPlex®.

A variety of immunoassays are known and may be used for the assays may be used in the present invention, these include: mass spectrometry (MS), luminescence immunoassay (LIA), radioimmunoassays (“RIA”), homogeneous enzyme-multiplied immunoassays (“EMIT”), enzyme linked immunoadsorbent assays (“ELISA”), apoenzyme reactivation immunoassay (“ARIS”), chemiluminescence—(“CLIA”), electrochemiluminescence—(“ECLIA”) and fluorescence-immunoassays, luminescence-based bead arrays, magnetic beads based arrays, protein microarray assays, rapid test formats such as for instance dipstick immunoassays, immuno-chromatographic strip tests, rare cryptate assay and automated systems/analyzers.

In one embodiment of the invention, it may be a so-called POC-test (point-of-care) that is a test technology, which allows performing the test within less than 1 hour near the patient without the requirement of a fully automated assay system. One example for this technology is the immunochromatographic test technology, e.g., a microfluidic device.

In a specific embodiment at least one of said two binders is labeled in said sandwich immunoassay in order to be detected.

In another preferred embodiment said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody.

The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassav Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence-based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in (Kirk-Othmer, Encyclopedia of chemical technology, 4th ed., executive editor, J I. Kroschwitz; editor, M Howe-Grant, John Wilev & Sons, 1993, vol. 15, p. 518-562, incorporated herein by reference, including citations on pages 551-562). Preferred chemiluminescent dyes are acridiniumesters.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

The ADM-NH2 μlevels of may be determined with the described ADM-NH2 assay (Weber et al. 2017. JALM 2(2):1-4). Other methods to quantify fragments derived from the ADM precursor have been described, e.g. the measurement of MR-proADM (Morgenthaler et al. 2005. Clin Chem 51(10):1823-9), PAMP (Washimine et al. 1994. Biochem Biophys Res Commun 202(2):1081-7) and CT-proADM (EP 2 111 552). A commercial homogeneous time-resolved fluoroimmunoassay for the measurement of MR-proADM in plasma on a fully automated system is available (BRAHMS MR-proADM KRYPTOR; BRAHMS GmbH, Hennigsdorf, Germany) (Caruhel et al. 2009. Clin Biochem 42(7-8):725-8). As these peptides are generated in a stoichiometric ratio from the same precursor, their plasma levels are correlated to a certain extent.

The above-mentioned threshold values might show a certain amount of variation, depending on the assay used to determine the level of the respective pro-ADM fragment, if these have been calibrated differently from the assay systems used in the present invention. Therefore, the above-mentioned thresholds shall apply in particular for the assay systems used in the present invention, and shall apply for such differently calibrated assays accordingly, taking into account the differences in calibration. One possibility of quantifying the difference in calibration is a method comparison analysis (correlation) of the assay in question (e.g. assay for measurement of proADM or fragments thereof) with the respective biomarker assay used in the present invention by measuring the respective biomarker (pro-ADM or a fragment thereof) in samples using both assays. Assuming a linear correlation between the assays, another possibility is to determine with the assay in question, given this test has sufficient analytical sensitivity, the median biomarker level of a representative normal population, compare results with the median biomarker levels as described in the literature (e.g. bio-ADM: Weber et al. 2017. J Appl Lab Med 2(2): 222-233; MR-proADM: Smith et al. 2009. Clin Chem 55:1593-1595; PAMP-Gly: Hashida et al. 2004. Clinical Biochemistry 37 14-21; mature PAMP: Hashida et al. 2004. Clinical Biochemistry 37 14-21) and recalculate the calibration based on the difference obtained by this comparison, e.g. by applying a factor. Hence, the x-fold of e.g. the mean or median (or a specific percentile) of the level of Pro-Adrenomedullin or a fragment thereof in a healthy population can be used as a threshold level using a differently calibrated assay, taking into account the above precautions.

The plasma median concentration of mature ADM (bio-ADM) in a normal (healthy) population was 13.7 μg/ml, the lowest value 11 μg/ml and the 99th percentile 43 μg/ml (Weber et al. 2017. J Appl Lab Med 2(2): 222-233).

A normal distribution of ADM-Gly from n=128 self-reported healthy individuals was determeind. The median concentration of ADM-Gly in this cohort was 27.1 μg/mL, the 25th percentile was 22 μg/mL, the 75th percentile was 32.7. pg/mL, the 5th percentile was 13.1 μg/mL, and the 95th percentile was 51.3 μg/mL.

The plasma median MR-proADM concentration in normal (healthy) subjects was 0.41 (interquartile range 0.23-0.64) nmol/L (Smith et al. 2009. Clin Chem 55:1593-1595) using the automated sandwich fluorescence assay for the detection of MR-proADM as described in Caruhel et al. (Caruhel et al. 2009. Clin Biochem 42:725-8).

The plasma median concentration of CT-proADM in normal healthy subjects (n=200) was 77.6 pmol/L (min 46.6 pmol/L, max 136.2 pmol/L) and the 950% percentile was 113.8 pmol/L (EP 2 111 552 B1).

The plasma mean concentration of PAMP-Gly in normal healthy subjects (n=51) was 1.15 pmol/L+/−0.38 pmol/L (Hashida et al. 2004. Clinical Biochemistry 37: 14-21).

The plasma mean concentration of mature PAMP in normal healthy subjects (n=51) was 0.51 pmol/L+/−0.19 pmol/L (Hashida et al. 2004. Clinical Biochemistry 37: 14-21).

A particular advantage of the invention is that patients can be stratified with respect to the required therapy, wherein said therapy is the administration of an anti-ADM antibody or anti-ADM fragment or anti-ADM non-Ig scaffold that binds to the N-terminal part (amino acid 1-21) of ADM: YRQSMNNFQGLRSFGCRFGTC (SEQ ID No.: 9). The stratified patient groups may include patients that require an initiation of treatment and patients that do not require initiation of treatment.

In a preferred embodiment, the treatment is initiated or changed immediately upon provision of the result of the sample analysis indicating the level of proADM or fragments thereof in the sample. In further embodiments, the treatment may be initiated within 12 hours, preferably 6, 4, 2, 1, 0.5, 0.25 hours or immediately after receiving the result of the sample analysis.

In some embodiments, the invention comprises or consists of a single and/or multiple measurement of proADM or fragments thereof in a sample from a patient in a single sample and/or multiple samples obtained at essentially the same time point, in order to guide and/or monitor and/or stratify a therapy, wherein said therapy is the administration of an anti-ADM antibody or anti-ADM fragment or anti-ADM non-Ig scaffold that binds to the N-terminal part (amino acid 1-21) of ADM: YRQSMNNFQGLRSFGCRFGTC (SEQ ID No.: 9).

In one embodiment of the invention described herein, the determined level of proADM or fragments thereof is compared to a reference and/or threshold level, wherein said comparing is carried out in a computer processor using computer executable code.

The comparison of the determined level of proADM or fragments thereof to a reference and/or threshold level may in part be computer-implemented. For example, the step of comparing the detected level of a marker, e.g., proADM or fragments thereof, with a reference and/or threshold level can be performed in a computer system. For example, the determined values may be entered (either manually by a health professional or automatically from the device(s) in which the respective marker level(s) has/have been determined) into the computer-system. The computer-system can be directly at the point-of-care (e.g., primary care unit or ED) or it can be at a remote location connected via a computer network (e.g., via the internet, or specialized medical cloud-systems, optionally combinable with other IT-systems or platforms such as hospital information systems (HIS)). Alternatively, or in addition, the associated therapy guidance and/or therapy stratification will be displayed and/or printed for the user (typically a health professional such as a physician).

Side effects of administration of immune effector cells in the frame of immune effector cell therapy are in particular selected from the group comprising cytokine release syndrome (CRS) and immune effector cell-associated neurotoxicity syndrome (ICANS).

In particular embodiments of the present invention, said immune effector cell therapy side effects are selected from the group comprising cytokine release syndrome (CRS), and immune effector cell-associated neurotoxicity syndrome (ICANS).

In more particular embodiments of the present invention, said cytokine release syndrome (CRS) or immune effector cell-associated neurotoxicity syndrome (ICANS) is mild (grade 1), moderate (grade 2), severe (3) or life-threatening (grade 4) or leads to death (grade 5).

In even more particular embodiments of the present invention, said immune effector cell therapy (IECT) is selected from the group comprising Chimeric Antigen Receptor T-cell (CAR-T) therapy, natural killer cell (NK) therapy, chimeric antigen receptor natural killer cell (CAR-NK) therapy, T cell receptor-engineered T cell (TCR T) therapy, tumor-infiltrating T cell (TIT)), and cytokine-induced killer cell (CIK) therapy.

In a specific embodiment of the invention said immune effector cell therapy is Chimeric Antigen Receptor T-cell (CAR T-cell) therapy.

For sake of completeness, this means that specific embodiments of the invention are:

Anti-ADM antibody or anti-ADM antibody fragment or anti-ADM non-Ig scaffold for use in therapy or prevention of side effects of CAR T-cell administration to a cancer patient in the frame of a Chimeric Antigen Receptor T-cell (CAR T-cell) therapy, wherein said anti-ADM antibody or anti-ADM antibody fragment or anti-ADM non-Ig scaffold is administered prior to or after administration of said immune effector cells to said patient.

Anti-ADM antibody or anti-ADM antibody fragment or anti-ADM non-Ig scaffold for use according to the invention, wherein said side effects of CAR T-cell administration in the frame of CAR T-cell therapy are associated with endothelial dysfunction.

Anti-ADM antibody or anti-ADM antibody fragment or anti-ADM non-Ig scaffold for use according to the invention, wherein said anti-ADM antibody or anti-ADM antibody fragment or anti-ADM non-Ig scaffold is used for reducing CAR T-cell therapy side effects. Anti-ADM antibody or anti-ADM antibody fragment or anti-ADM non-Ig scaffold for use according to the invention, wherein said CAR T-cell therapy side effects are selected from the group comprising cytokine release syndrome (CRS), and immune effector cell-associated neurotoxicity syndrome (ICANS).

Anti-ADM antibody or anti-ADM antibody fragment or anti-ADM non-Ig scaffold for use according to the invention, wherein said therapy comprises the administration of further medication for the treatment of CAR T-cell therapy side effects, particularly selected from the group comprising antihistamines, non-steroidal anti-inflammatory drugs (NSAIDs), immunosuppressive drugs, vasopressors, fluids and oxygen, more particularly wherein said immunosuppressive drugs are one or more drugs selected from the group comprising anti-IL-6 receptor antibody (e.g. tocilizumab), anti-IL-6 antibody (e.g. siltuximab), anti-TNF antibody (e.g. etanercept, infliximab), anti-IL-1 antibody (e.g. anakinra) and corticosteroids, and/or wherein said corticosteroid is selected from the group comprising cortisone, hydrocortisone, fludrocortisone, dexamethasone, betamethasone, prednisolone, methylprednisolone, and prednisone.

For sake of completeness, the further embodiments of the invention likewise read on the specific embodiments relating to CAR T-cell therapy, such as in particular embodiment relating to levels of Pro-ADM or fragments thereof, particularly Pro-ADM, mature PAMP, PAMP-Gly, MR-proADM, ADM-Gly, mature ADM, CT-proADM, in a sample of bodily fluid above a threshold, more particularly the specific threshold levels of Pro-ADM or fragments thereof disclosed herein, specific samples of bodily fluid, as well as thr anti-ADM antibodys, anti-ADM antibody fragments or anti-ADM non-Ig scaffolds binding to specific sequences and/or comprising specific sequences as disclosed herein, in particular binding to SEQ ID No.: 9 and/or having a heavy chain comprising SEQ ID Nos.: 19-21 and a light chain comprises the SEQ ID No.: 22, RVS and SEQ ID No.: 23, or comprising as a heavy chain SEQ ID No.: 32 or a sequence that is >95% identical to it, and comprising as a light chain SEQ ID No.: 33 or a sequence that is >95% identical to it, or comprising a heavy chain Fab Fragment comprising or variable region portion SEQ ID NO: 35 and/or a light chain Fab Fragment comprising or variable region portion SEQ ID NO: 33.

In particular embodiments of the present invention, the therapy comprises the administration of further medication for the treatment of immune effector cell therapy side effects.

In a specific embodiment of the invention, said further medication is selected from the group comprising antihistamins, non-steroidal anti-inflammatory drugs (NSAIDs), immunosuppressive drugs, vasopressors, fluids and oxygen.

In a more specific embodiment of the invention, said immune effector cell therapy side effects are in addition treated with one or more immunosuppressive drugs selected from the group comprising anti-IL-6 receptor antibody (e.g. tocilizumab), anti-IL-6 antibody (e.g. siltuximab), anti-TNF antibody (e.g. etanercept, infliximab), anti-IL-1 antibody (e.g. anakinra) and corticosteroids.

In an even more specific embodiment, corticosteroids are selected from the group consisting of cortisone, hydrocortisone, fludrocortisone, dexamethasone, betamethasone, prednisolone, methylprednisolone, and prednisone.

### Anti-ADM Antibody/Antibody Fragment/Non-Ig Scaffold

In more particular embodiments of the present invention, said anti-ADM antibody or anti-ADM fragment or anti-ADM non-Ig scaffold binds to the N-terminal part (amino acids 1-21) of ADM:

(SEQ ID No.: 9)
YRQSMNNFQGLRSFGCRFGTC.

In more particular embodiments of the present invention, said anti-ADM antibody or anti-ADM antibody fragment or anti-ADM non-Ig scaffold recognizes and binds to the N-terminal moiety (comprising amino acid 1) of ADM.

In more particular embodiments of the present invention, said antibody, antibody fragment or non-Ig scaffold does not bind to the C-terminal portion of ADM, having the sequence amino acid 43-52 of ADM: PRSKISPQGY-NH₂ (SEQ ID No.: 15).

In even more particular embodiments of the present invention, said antibody or fragment is a monoclonal antibody or fragment that binds to ADM or an antibody fragment thereof, wherein the heavy chain comprises the sequences:

CDR1:
SEQ ID No.: 19
GYTFSRYW
CDR2:
SEQ ID No.: 20
ILPGSGST
CDR3:
SEQ ID No.: 21
TEGYEYDGFDY

and wherein the light chain comprises the sequences:

CDR1:
SEQ ID No.: 22
QSIVYSNGNTY
CDR2:
RVS
CDR3:
SEQ ID No.: 23
FQGSHIPYT.

In even more particular embodiments of the present invention, said antibody or fragment comprises a sequence selected from the group comprising as a VH region:

(AM-VH-C)
SEQ ID No.: 24
QVQLQQSGAELMKPGASVKISCKATGYTFSRYWIEWVKQRPGHGLEWIGEILPGSGSTNYNE
KFKGKATITADTSSNTAYMQLSSLTSEDSAVYYCTEGYEYDGFDYWGQGTTLTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKRVEPK
(AM-VH1)
SEQ ID No.: 25
QVQLVQSGAEVKKPGSSVKVSCKASGYTFSRYWISWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH2-E40)
SEQ ID No.: 26
QVQLVQSGAEVKKPGSSVKVSCKASGYTFSRYWIEWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH3-T26-E55)
SEQ ID No.: 27
QVQLVQSGAEVKKPGSSVKVSCKATGYTFSRYWISWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH4-T26-E40-E55)
SEQ ID No.: 28
QVQLVQSGAEVKKPGSSVKVSCKATGYTFSRYWIEWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK

and comprises a sequence selected from the group comprising the following sequence as a VL region:

(AM-VL-C)
SEQ ID No.: 29
DVLLSQTPLSLPVSLGDQATISCRSSQSIVYSNGNTYLEWYLQKPGQSPKLLIYRVSNRFSGVP
DRFSGSGSGTDFTLKISRVEAEDLGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
(AM-VL1)
SEQ ID No.: 30
DVVMTQSPLSLPVTLGQPASISCRSSQSIVYSNGNTYLNWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
(AM-VL2-E40)
SEQ ID No.: 31
DVVMTQSPLSLPVTLGQPASISCRSSQSIVYSNGNTYLEWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC.

In an even more particular embodiment of the present invention, said antibody or fragment comprises the following sequence as a heavy chain:

SEQ ID No.: 32
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

or a sequence that is >95% identical to it,
and comprises the following sequence as a light chain:

SEQ ID No.: 33
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC

or a sequence that is >95% identical to it.

The efficacy of non-neutralizing antibody targeted against the N-terminus of ADM was investigated in a survival study in CLP-induced sepsis in mice. Pre-treatment with the non-neutralizing antibody resulted in decreased catecholamine infusion rates, kidney dysfunction, and ultimately improved survival (Struck et al. 2013. Intensive Care Med Exp 1(1):22, Wagner et al. 2013. Intensive Care Med Exp1(1):21).

Due to these positive results, a humanized version of an N-terminal anti-ADM antibody, named Enibarcimab, has been developed for further clinical development. Beneficial effects of Enibarcimab on vascular barrier function and survival were recently demonstrated in preclinical models of systemic inflammation and sepsis (Geven et al. 2018. Shock 50(6):648-654). In this study, pre-treatment with Enibarcimab attenuated renal vascular leakage in endotoxemic rats as well as in mice with CLP-induced sepsis, which coincided with increased renal expression of the protective peptide Ang-1 and reduced expression of the detrimental peptide vascular endothelial growth factor. Also, pre-treatment with Enibarcimab improved 7-day survival in CLP-induced sepsis in mice from 10 to 50% for single and from 0 to 40% for repeated dose administration. Moreover, in a phase I study, excellent safety and tolerability was demonstrated (see Example 4): no serious adverse events were observed, no signal of adverse events occurring more frequently in Enibarcimab-treated subjects was detected and no relevant changes in other safety parameters were found (Geven et al. 2017. Intensive Care Med Exp 5 (Suppl 2): 0427). Of particular interest is the proposed mechanism of action of Enibarcimab. Both, animal and human data reveal a potent, dose-dependent increase of circulating ADM following administration of this antibody. Based on pharmacokinetic data and the lack of an increase in MR-proADM (an inactive peptide fragment derived from the same prohormone as ADM), the higher circulating ADM levels cannot be explained by an increased production.

A mechanistic explanation for this increase could be that the excess of antibody in the circulation may drain ADM from the interstitium to the circulation, since ADM is small enough to cross the endothelial barrier, whereas the antibody is not (Geven et al. 2018. Shock. 50(2):132-140). In addition, binding of the antibody to ADM leads to a prolongation of ADM's half-life. Even though NT-ADM antibodies partially inhibit ADM-mediated signalling, a large increase of circulating ADM results in an overall “net” increase of ADM activity in the blood compartment, where it exerts beneficial effects on endothelial cells (ECs; predominantly barrier stabilization), whereas ADMs detrimental effects on vascular smooth muscle cells (VSMCs; vasodilation) in the interstitium are reduced.

Throughout the specification the “antibodies”, or “antibody fragments” or “non-Ig scaffolds” in accordance with the invention are capable to bind ADM, and thus are directed against ADM, and thus can be referred to as “anti-ADM antibodies”, “anti-ADM antibody fragments”, or “anti-ADM non-Ig scaffolds”.

The term “antibody” generally comprises monoclonal and polyclonal antibodies and binding fragments thereof, in particular Fc-fragments as well as so called “single-chain-antibodies” (Bird et al. 1988), chimeric, humanized, in particular CDR-grafted antibodies, and dia or tetrabodies (Holliger et al. 1993). Also comprised are immunoglobulin-like proteins that are selected through techniques including, for example, phage display to specifically bind to the molecule of interest contained in a sample. In this context the term “specific binding” refers to antibodies raised against the molecule of interest or a fragment thereof.

An antibody is considered to be specific, if its affinity towards the molecule of interest or the aforementioned fragment thereof is at least preferably 50-fold higher, more preferably 100-fold higher, most preferably at least 1000-fold higher than towards other molecules comprised in a sample containing the molecule of interest. It is well known in the art how to make antibodies and to select antibodies with a given specificity.

In one embodiment of the invention the anti-Adrenomedullin (ADM) antibody or anti-adrenomedullin antibody fragment or anti-ADM non-Ig scaffold is monospecific.

Monospecific anti-adrenomedullin (ADM) antibody or monospecific anti-adrenomedullin antibody fragment or monospecific anti-ADM non-Ig scaffold means that said antibody or antibody fragment or non-Ig scaffold binds to one specific region encompassing at least 5 amino acids within the target ADM. Monospecific anti-Adrenomedullin (ADM) antibody or monospecific anti-adrenomedullin antibody fragment or monospecific anti-ADM non-Ig scaffold are anti-adrenomedullin (ADM) antibodies or anti-adrenomedullin antibody fragments or anti-ADM non-Ig scaffolds that all have affinity for the same antigen. Monoclonal antibodies are monospecific, but monospecific antibodies may also be produced by other means than producing them from a common germ cell.

Said anti-ADM antibody or antibody fragment binding to ADM or non-Ig scaffold binding to ADM may be a non-neutralizing anti-ADM antibody or antibody fragment binding to ADM or non-Ig scaffold binding to ADM.

In a specific embodiment said anti-ADM antibody, anti-ADM antibody fragment or anti-ADM non-Ig scaffold is a non-neutralizing antibody, fragment or non-Ig scaffold. A neutralizing anti-ADM antibody, anti-ADM antibody fragment or anti-ADM non-Ig scaffold would block the bioactivity of ADM to nearly 100%, to at least more than 90%, preferably to at least more than 95%.

In contrast, a non-neutralizing anti-ADM antibody, or anti-ADM antibody fragment or anti-ADM non-Ig scaffold blocks the bioactivity of ADM less than 100%, preferably to less than 95%, preferably to less than 90%, more preferred to less than 80% and even more preferred to less than 50%. This means that bioactivity of ADM is reduced to less than 100%, to 95% or less but not more, to 90% or less but not more, to 80% or less but not more, to 50% or less but not more. This means that the residual bioactivity of ADM bound to the non-neutralizing anti-ADM antibody, or anti-ADM antibody fragment or anti-ADM non-Ig scaffold would be more than 0%, preferably more than 5%, preferably more than 10%, more preferred more than 20%, more preferred more than 50%.

In this context (a) molecule(s), being it an antibody, or an antibody fragment or a non-Ig scaffold with “non-neutralizing anti-ADM activity”, collectively termed here for simplicity as “non-neutralizing” anti-ADM antibody, antibody fragment, or non-Ig scaffold, that e.g. blocks the bioactivity of ADM to less than 80%, is defined as
a molecule or molecules binding to ADM, which upon addition to a culture of an eukaryotic cell line, which expresses functional human recombinant ADM receptor composed of CRLR (calcitonin receptor like receptor) and RAMP3 (receptor-activity modifying protein 3), reduces the amount of cAMP produced by the cell line through the action of parallel added human synthetic ADM peptide, wherein said added human synthetic ADM is added in an amount that in the absence of the non-neutralizing antibody to be analyzed, leads to half-maximal stimulation of cAMP synthesis, wherein the reduction of cAMP by said molecule(s) binding to ADM takes place to an extent, which is not more than 80%, even when the non-neutralizing molecule(s) binding to ADM to be analyzed is added in an amount, which is 10-fold more than the amount, which is needed to obtain the maximal reduction of cAMP synthesis obtainable with the non-neutralizing antibody to be analyzed.

The same definition applies to the other ranges; 95%, 90%, 50% etc.

An antibody or fragment according to the present invention is a protein including one or more polypeptides substantially encoded by immunoglobulin genes that specifically binds an antigen. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length.

Full-length immunoglobulin heavy chains are generally about 50 Kd or 446 amino acid in length. Light chains are encoded by a variable region gene at the NH2-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al. 1987. Eur. J. Immunol. 17:105; Huston et al. 1988. Proc. Natl. Acad. Sci. U.S.A., 85:5879-5883; Bird et al. 1988. Science 242:423-426; Hood et al. 1984, Immunology, Benjamin, N.Y, 2nd ed.; Hunkapiller and Hood 1986. Nature 323:15-16). An immunoglobulin light or heavy chain variable region includes a framework region interrupted by three hypervariable regions, also called complementarity determining regions (CDR's) (see, Sequences ofProteins ofImmunological Interest, E. Kabat et al. 1983, U.S. Department ofHealth and Human Services). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDR's. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr.

Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection of a B-cell or by fusing a B cell with a myeloma to make a hybridoma or fusing a B cell to a hybridoma cell to produce a trioma cell. Human antibodies can also be produced by phage display methods (see, e.g. WO91/17271; WO92/001047; WO92/20791), or selected from a human combinatorial monoclonal antibody library (see the Morphosys website). Human antibodies can also be prepared by using transgenic animals carrying a human immunoglobulin gene (for example, see WO93/12227; WO 91/10741).

Thus, the anti-ADM antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)₂-fragments, scFv-fragments, multimerized multivalent or/and multi-specific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines and numerous others.

In addition to anti-ADM antibodies other biopolymer scaffolds are well known in the art to complex a target molecule and have been used for the generation of highly target specific biopolymers. Examples are aptamers, spiegelmers, anticalins and conotoxins. For illustration of antibody formats please see FIGS. 1A, 1B and 1C.

In a preferred embodiment the anti-ADM antibody format is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is the scFab format.

Non-Ig scaffolds may be protein scaffolds and may be used as antibody mimics as they are capable to bind to ligands or antigens. Non-Ig scaffolds may be selected from the group comprising tetranectin-based non-Ig scaffolds (e.g. described in US 2010 0028995), fibronectin scaffolds (e.g. described in EP 1 266 025; lipocalin-based scaffolds (e.g. described in WO 2011 154420); ubiquitin scaffolds (e.g. described in WO 2011 073214), transferrin scaffolds (e.g. described in US 2004 0023334), protein A scaffolds (e.g. described in EP 2 231 860), ankyrin repeat based scaffolds (e.g. described in WO 2010 060748), microproteins preferably microproteins forming a cysteine knot) scaffolds (e.g. described in EP 2314308), Fyn SH3 domain based scaffolds (e.g. described in WO 2011 023685) EGFR-A-domain based scaffolds (e.g. described in WO 2005 040229) and Kunitz domain based scaffolds (e.g. described in EP 1941 867.

In one embodiment of the invention anti-ADM antibodies according to the present invention may be produced as outlined in Example 1 by synthesizing fragments of ADM as antigens. Thereafter, binder to said fragments are identified using the below described methods or other methods as known in the art.

### Methods for Obtaining Monoclonal Antibodies

In all of the following embodiments, the term monoclonal antibody is meant to include monoclonal antibodies, as well as fragments of monoclonal antibodies, such as the ones detailed herein, more particularly monoclonal antibodies.

### Hybridoma

In a further aspect, the antibody according to the present invention is a monoclonal antibody obtainable by a method comprising:
i) fusing antibody-secreting cells from an animal previously immunized with an antigen with myeloma cells to obtain a multitude of hybridomas,
ii) isolating from said multitude of hybridomas a hybridoma producing a desired monoclonal antibody.

In certain embodiments, the antibody according to the present invention is a monoclonal antibody obtainable by isolating from a multitude of hybridomas a hybridoma producing a desired monoclonal antibody, wherein said multitude of hybridomas were produced by fusing antibody-secreting cells from an animal previously immunized with an antigen with myeloma cells to obtain multitude of hybridomas.

A desired monoclonal antibody is in particular a monoclonal antibody binding the antigen, in particular with a binding affinity of at least 10⁷ M⁻¹, preferred 10⁸ M⁻¹, more preferred affinity is greater than 10⁹ M⁻¹, most preferred greater than 10¹⁰ M⁻¹.

In certain embodiments of the method for obtaining an antibody, in step i) the animal is a mammal, particularly a rabbit, a mouse or a rat, more particularly a mouse, more particularly a Balb/c mouse.

In certain embodiments of the method for obtaining an antibody, in step i) the antibody-secreting cell is a splenocyte, more particularly an activated B-cell.

In certain embodiments of the method for obtaining an antibody, in step i) fusing involves the use of polyethylene glycol.

In certain embodiments of the method for obtaining an antibody, in step i) the myeloma is derived from a mammal, in certain embodiments from the same species of mammal from which the multitude of antibody-secreting cells is obtained. In certain specific embodiments of the method for obtaining an antibody, in step i) the myeloma cells are of the cell line SP2/0.

In certain embodiments of the method for obtaining an antibody, said fusing in step i) comprises PEG-assisted fusion, Sendai virus-assisted fusion or electric current-assisted fusion.

In certain embodiments of the method for obtaining an antibody, said isolating in step ii) comprises performing an antibody capture assay, an antigen capture assay, and/or a functional screen.

In certain embodiments of the method for obtaining an antibody, in step ii) isolating the hybridoma producing a desired monoclonal antibody may involve cloning and re-cloning the hybridomas using the limiting-dilution technique.

In one embodiment, said antigen capture assay comprises:
a) binding the produced antibodies to a substrate, particularly a solid substrate, b) allowing antigen to bind to said antibodies,
c) removing unbound antigen by washing,
d) detecting bound antigen;
or said antigen capture assay comprises:
a) allowing an antigen to bind the produced antibodies to form an antibody-antigen complex,
b) binding said antibody-antigen complex to a substrate, particularly a solid substrate,
c) removing unbound antigen by washing,
d) detecting bound antigen.

In one embodiment, said isolating of step ii) comprises performing an enzyme-linked immunosorbent assay, fluorescence-activated cell sorting, cell staining, immunoprecipitation, and/or a western blot. In one embodiment, said detecting of the antibody or the antigen is accomplished with an immunoassay. In one embodiment, the animal is a transgenic animal, in particular a transgenic mouse (wherein in particular the mouse immunoglobulin (Ig) gene loci have been replaced with human loci within the transgenic animal genome), such as HuMabMouse or XenoMouse.

In one embodiment, the antigen comprises a peptide as described herein in Table 1, which in certain embodiments (in particular for immunization) may be conjugated to a protein, particularly a serum protein, more particularly a serum albumin, more particularly BSA.

In a preferred embodiment, the antibody according to the present invention is a monoclonal antibody obtainable by a method comprising:
i) fusing splenocytes cells from a Balb/c mouse previously immunized with a peptide as described herein in Table 1 with SP2/0 myeloma cells using polyethylene glycol, to obtain a multitude of hybridomas,
ii) isolating from said multitude of hybridomas a hybridoma producing a desired monoclonal antibody;
   more preferably, the method comprises:
1) growing hybridomas for a first period (in particular 2 weeks) in HAT medium [RPMI 1640 culture medium supplemented with 20% fetal calf serum and HAT-Supplement]
2) followed replacing HAT medium with HT Medium for a multitude of passages (in particular 3)
3) followed by returning to the normal cell culture medium for a second time period, in particular until the end of three weeks after fusion
4) primary screening of cell culture supernatants for antigen-specific IgG antibodies
5) propagating microcultures of cells that tested positive in 4)
6) retesting cell culture supernatants of microcultures for antigen-specific IgG antibodies
7) cloning and re-cloning cultures that tested positive in 6), using the limiting-dilution technique
8) optionally determining the isotypes of clones obtained from 7)
9) optionally purifying antibodies via Protein A

### Phage Display

In a further aspect, the antibody according to the present invention is a monoclonal antibody obtainable by a method comprising:
i) isolating at least one antibody having affinity to an antigen from an antibody gene library;
ii) generating at least one cell strain expressing said at least one antibody;
iii) isolating the at least one antibody from a culture of the at least one cell strain obtained in step ii).

An antibody having affinity to an antigen is in particular an antibody with a binding affinity of at least 10⁷ M⁻¹, preferred 10⁸ M⁻¹, more preferred affinity is greater than 10⁹ M⁻¹, most preferred greater than 10¹⁰ M⁻¹.

In a certain embodiment, the antibody according to the present invention is a monoclonal antibody obtainable by isolating at least one antibody from a culture derived from at least one cell strain which expressed at least one antibody having affinity to an antigen from an antibody gene library.

In one embodiment, the antigen comprises a peptide as described herein in Table 1, which in certain embodiments may be bound to a solid phase.

In certain embodiments of the method for obtaining an antibody, in step i) the antibody gene library is a naive antibody gene library, particularly a human naive antibody gene library, more particularly in said library the antibodies are presented via phage display, i.e. on phages comprising a nucleotide sequence encoding for such respective antibody; more particularly the library HAL 7, HAL 8, or HAL 9, more particularly a library comprising the human naive antibody gene libraries HAL7/8.

In certain embodiments of the method for obtaining an antibody, in step i) screening comprises the use of an antigen, particularly an antigen containing a tag, more particularly a biotin tag, linked thereto via two different spacers. In particular embodiments, such panning strategy includes a mix of panning rounds with non-specifically bound antigen and antigen bound specifically via the tag, in the case of a biotin tag, bound to streptavidin. In this way, the background of non-specific binders may be minimized.

In certain embodiments of the method for obtaining an antibody, in step i), in embodiments wherein the library is a phage display library, the antibody is isolated by isolating a phage presenting said antibody (and comprising a nucleotide sequence encoding for the antibody).

In certain embodiments of the method for obtaining an antibody, in step ii) said cell strain is generated via introduction of a nucleotide sequence encoding for the antibody), in embodiments wherein the library in step i) is a phage display library, the isolated phage from step i) may be used to produce a bacterial strain, e.g. an E. coli strain, expressing the antibody.

In certain embodiments of the method for obtaining an antibody, in step iv); in embodiments wherein the library in step i) is a phage display library and wherein a bacterial strain is produced in step ii), antibody may be isolated from the supernatant of the culture.

It is understood that, as used in describing the methods for obtaining an antibody, the term “one antibody” in the expression “at least one antibody” in particular may include more than one antibody molecule of antibodies having the same amino acid sequence. This understanding applies, mutatis mutandis, to the term “one cell strain”.

In certain embodiments of the method for obtaining an antibody, more than one antibody (referring to a multitude of antibodies having distinct amino acid sequences, respectively) is isolated in step i) and accordingly more than one cell strain is generated in step ii). Such method may involve the selection of clones that are positive for binding to the antigen, e.g. via a binding assay, e.g. an ELISA assay involving the antigen, and cells positive for binding to the antigen may be isolated to produce monoclonal cell strains.

In a preferred embodiment, the antibody according to the present invention is a monoclonal antibody obtainable by a method comprising:
i) isolating at least one antibody having affinity to an antigen from an antibody gene library comprising the human naive antibody gene libraries HAL7/8, by eluting phages carrying said antibody from the library;
ii) generating at least one E. coli cell strain expressing said at least one antibody;
iii) isolating the at least one antibody from the supernatant a culture of the at least one E. coli cell strain obtained in step ii).

In a further aspect, an antibody fragment according to the present invention is produced by a method in volving enzymatic digestion of an antibody.

In certain embodiments, this method produces e.g. Fab or F(ab)₂ antibody fragments. In certain embodiments, this method involves digestion with pepsin or papain, which are optionally immobilized on a surface.

In certain embodiments, antibodies may be humanized by CDR-grafting, in particular by a process involving the steps:
extracting RNA from hybridomas expressing an antibody of interest (e.g. obtained by a method as described herein);
amplifying said extracted RNA via RT-PCR, in particular with primer sets specific for the heavy and light chains of the antibody of interest, to obtain to obtain a DNA product;
further amplifying said DNA product via PCR, in particular using semi-nested primer sets specific for antibody variable regions;
determining the sequence of the DNA product;
aligning said sequence with homologous human framework sequences to determine a humanized sequence for the variable heavy chain and the variable light chain sequences (of the desired antibody).

In certain embodiments, antibodies may be humanized by aligning the sequence of a DNA product that was obtained by amplifying RNA extracted from hybridomas expressing an antibody of interest via RT-PCR, in particular with primer sets specific for the heavy and light chains of the antibody of interest and further amplifying the DNA obtained therefrom via PCR, in particular using semi-nested primer sets specific for antibody variable regions, with homologous human framework sequences to determine a humanized sequence for the variable heavy chain and the variable light chain sequences (of the desired antibody).

In certain embodiments, antibodies may be humanized by
determining the complementary determining regions (CDR), which may be accomplished by analysing the structural interaction of framework regions (FR) with the complementary determining regions (CDR) and the antigen;
transplanting said CDR sequences into a human framework region.

In certain embodiments, antibodies may be humanized by transplanting CDR sequences, which may preferably have been determined by analysing the structural interaction of framework regions (FR) with the complementary determining regions (CDR) and the antigen, into a human framework region.

In certain embodiments variations in the amino acid sequence of the CDRs or FRs may be introduced to maintain structural interactions with the antigen (which may otherwise be abolished by introducing the human FR sequences), for instance by a random approach using phage display libraries or via directed approach guided by molecular modelling.

The DNA sequences encoding for antibodies determined as detailed herein can be transferred by known genetic engineering techniques into cells and used for production of the antibody.

### Producing Antibodies

In a further aspect, the antibody according to the present invention is a monoclonal antibody obtainable by the methods described herein, produced by a method comprising:
culturing a cell strain comprising a nucleotide sequence encoding for the antibody;
isolating the antibody from said culture.

In a further certain aspect, the antibody according to the present invention is a monoclonal antibody obtainable by the methods described herein, produced by isolating the antibody from a culture of a cell strain comprising a nucleotide sequence encoding for said antibody.

In certain embodiments of said method, the cell strain is produced as described herein above and may comprise bacterial cells, such as gram-negative bacteria, e.g. E. coli, Proteus mirabilis, or Pseudomonas putidas, gram-positive bacteria, e.g. Bacillus brevis, Bacillus subtilis, Bacillus megaterium, Lactobacilli such as Lactobacillus zeae casei or Lactobacillus paracasei, or Streptomyces, such as Streptomyces lividans; eucariotic cells such as yeast, e.g. Pichia pastoris, Saccharomyces cerevisiae, Hansenula polymorpha, Schizosaccharomyces pombe, Schwanniomyces occidentalis, Kluyveromyces lactis, or Yarrowia lipolytica; fugi, such as filamentous fungi, e.g. of the genus Trichoderma of Aspergillus, such as A. niger (e.g. subgenus A. awamori) and Aspergillus oryzae, Trichoderma reesei, Chrysosporium, such as C. lucknowense; protozoae, such as Leishmania, e.g. L. tarentolae; insect cells, such as insect cells transfected a Baculovirus, e.g. AcNPV, such as insect cell lines from Spodopterafrugiperda, e.g. Sf-9 or Sf-21, Drosophila melanogaster, e.g. DS2, or Trichopulsia ni, e.g. High Five cells (BTI-TN-5B1-4); mammalian cells such as hamster, e.g. Chinese hamster ovary such as K1-, DukX B 11-, DG44, Lec13, or BHK, mouse, e.g. mouse myeloma such as NS0, Homo sapiens, e.g. Per.C6, AGE1.HN, HEK293.

In certain embodiments of said method, the cells may be hybridoma cells, e.g. as described herein.

In certain embodiments of said method, culturing may take place in a static suspension culture, an agitated suspension culture, a membrane-based culture, a matrix-based culture or a high cell density bioreactor; a vessel for such culturing may be selected from the group comprising a T-flask, a roller culture, a spinner culture, a stirred tank bioreactor, an airlift bioreactor, a static membrane-based or matrix-based culture system, a suspension bioreactor, a fluidized bed bioreactor, a ceramic bioreactor, a perfusion system, a hollow fiber bioreactor.

In certain embodiments of said method, the cells may be immobilized on a matrix.

A high cell density bioreactor is in particular a culture system capable of generating cell densities greater than 10⁸ cells/ml.

In a further aspect, the antibody according to the present invention is a monoclonal antibody obtainable by the methods described herein, produced by a method comprising:
generating a transgenic plant or animal comprising a nucleotide sequence encoding for the antibody;
isolating the antibody from said plant or animal or a secretion or product of said plant or animal.

In a certain further aspect, the antibody according to the present invention is a monoclonal antibody obtainable by the methods described herein, produced by isolating the antibody from a transgenic plant or transgenic animal or a secretion or product of a transgenic plant or transgenic animal having a nucleotide sequence encoding for the antibody.

Said animal may e.g., be selected from a chicken, a mouse, a rat, a rabbit, a cow, a goat, a sheep, a pig; said secretion or product may e.g. be milk or an egg. Said plant may e.g. be selected from tobacco (N. tabacum or N. benthamiana), duckweed (Lemna minor), Chlamydomonas reinhardtii, rice, Arabidopsis thaliana, alfalfa (Medicago sativa), lettuce, maize.

The antibodies can in certain embodiments be isolated by physicochemical fractionation, e.g. size exclusion chromatography, precipitation, e.g. using ammonium sulphate, ion exchange chromatography, immobilized metal chelate chromatography gel filtration, zone electrophoresis; based on their classification e.g. binding to bacterial proteins A, G, or L, jacalin; antigen-specific affinity purification via immobilized ligands/antigens; if necessary, low molecular weight components can be removed by methods like dialysis, desalting, and diafiltration.

In some embodiments the antibody is encoded by a nucleotide sequence where the nucleotide sequence is a reverse transcription of an amino acid sequence from an antibody produced by one of the processes described herein.

Humanization of murine antibodies may be conducted according to the following procedure:

For humanization of an antibody of murine origin the antibody sequence is analyzed for the structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modelling (Almagro 2008) The use of molecular modeling to achieve a humanized antibodies may involve additional conservative amino acid substitutions at the junction of the CDR-FR. Exemplary conservative substitutions are those such as gly, ala; val, ile, leu; asp, glu; asn, gln; ser, thr; lys, arg; and phe, tyr. Additionally, back mutations may be implemented, where selection of residues outside of the region defining the specificity and thus target for back mutation (to mimic the source sequence, i.e., murine) to restore or improve the affinity of the humanized antibody. (Almagro 2008). When available, the three-dimensional models of the antibodies may be used to guide the humanization process.

In a preferred embodiment the ADM antibody format is selected from the group comprising Fv fragment, scFv fragment, Fab fragment, scFab fragment, F(ab)₂ fragment and

scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is scFab format.

In another preferred embodiment, the anti-ADM antibody, anti-ADM antibody fragment, or anti-ADM non-Ig scaffold is a full-length antibody, antibody fragment, or non-Ig scaffold.

In a preferred embodiment the anti-ADM antibody or an anti-ADM antibody fragment or anti-ADM non-Ig scaffold is directed to and can bind to an epitope of at least 5 amino acids in length contained in ADM.

In a more preferred embodiment, the anti-ADM antibody or an anti-ADM antibody fragment or anti-ADM non-Ig scaffold is directed to and can bind to an epitope of at least 4 amino acids in length contained in ADM.

In one specific embodiment of the invention the anti-ADM antibody or anti-ADM antibody fragment binding to adrenomedullin or anti-ADM non-Ig scaffold binding to adrenomedullin is provided for use in therapy or prevention according to the present invention, wherein said antibody or fragment or scaffold is not ADM-binding-Protein-1 (complement factor H).

In one specific embodiment of the invention the anti-Adrenomedullin (ADM) antibody or anti-ADM antibody fragment binding to adrenomedullin or anti-ADM non-Ig scaffold binding to adrenomedullin is provided for use in therapy or prevention according to the present invention, wherein said antibody or fragment or scaffold binds to a region of preferably at least 4, or at least 5 amino acids within the sequence of amino acid 1-21 of mature human ADM: YRQSMNNFQGLRSFGCRFGTC (SEQ ID No.: 9).

In a preferred embodiment of the present invention said anti-ADM antibody or anti-adrenomedullin antibody fragment or anti-ADM non-Ig scaffold binds to a region or epitope of ADM that is located in the N-terminal part (amino acid 1-21) of adrenomedullin.

In another preferred embodiment said anti-ADM-antibody or anti-ADM antibody fragment or anti-ADM non-Ig scaffold recognizes and binds to a region or epitope within amino acids 1-14 of adrenomedullin: YRQSMNNFQGLRSF (SEQ ID No.: 16) that means to the N-terminal part (amino acid 1-14) of adrenomedullin.

In another preferred embodiment said anti-ADM-antibody or anti-adrenomedullin antibody fragment or anti-ADM non-Ig scaffold recognizes and binds to a region or epitope within amino acids 1-10 of adrenomedullin: YRQSMNNFQG (SEQ ID No.: 17); that means to the N-terminal part (amino acid 1-10) of adrenomedullin.

In another preferred embodiment said anti-ADM antibody or anti-ADM antibody fragment or anti-ADM non-Ig scaffold recognizes and binds to a region or epitope within amino acids 1-6 of adrenomedullin: YRQSMN (SEQ ID No.: 18); that means to the N-terminal part (amino acid 1-6) of adrenomedullin. As stated above said region or epitope comprises preferably at least 4 or at least 5 amino acids in length.

In another preferred embodiment said anti-ADM antibody or anti-ADM antibody fragment or anti-ADM non-Ig scaffold recognizes and binds to the N-terminal end (amino acid 1) of adrenomedullin. N-terminal end means that the amino acid 1, that is “Y” of SEQ ID No.s 16, 17 or 18, respectively and is mandatory for binding. The antibody or fragment or scaffold would neither bind N-terminal extended nor N-terminal modified Adrenomedullin nor N-terminal degraded adrenomedullin. This means in another preferred embodiment said anti-ADM-antibody or anti-ADM antibody fragment or anti-ADM non-Ig scaffold binds only to a region within the sequence of mature ADM if the N-terminal end of ADM is free. In said embodiment the anti-ADM antibody or anti-ADM antibody fragment or non-Ig scaffold would not bind to a region within the sequence of mature ADM if said sequence is e.g. comprised within pro-ADM.

For the sake of clarity, the numbers in brackets for specific regions of ADM like “N-terminal part (amino acid 1-21)” is understood by a person skilled in the art that the N-terminal part of ADM consists of amino acids 1-21 of the mature ADM sequence.

In another specific embodiment pursuant to the invention the herein provided anti-ADM antibody or anti-ADM antibody fragment or anti-ADM non-Ig scaffold does not bind to the C-terminal portion of ADM, i.e., the amino acid 43-52 of ADM: PRSKISPQGY-NH₂ (SEQ ID No.: 15).

An epitope, also known as antigenic determinant, is the part of an antigen that is recognized by the immune system, specifically by antibodies. For example, the epitope is the specific piece of the antigen to which an antibody binds. The part of an antibody that binds to the epitope is called a paratope. The epitopes of protein antigens are divided into two categories, conformational epitopes and linear epitopes, based on their structure and interaction with the paratope.

Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontiguous amino acid residues. A linear or a sequential epitope is an epitope that is recognized by antibodies by its linear sequence of amino acids, or primary structure and is formed by the 3-D conformation adopted by the interaction of contiguous amino acid residues.

In one specific embodiment it is preferred to use an anti-ADM antibody or anti-ADM antibody fragment or anti-ADM non-Ig scaffold according to the present invention, wherein said anti-ADM antibody or said anti-ADM antibody fragment or anti-ADM non-Ig scaffold leads to an increase of the ADM level or ADM immunoreactivity in serum, blood, plasma of at least 10%, preferably at least 50%, more preferably >50%, most preferably >100%.

In one specific embodiment it is preferred to use an anti-ADM antibody or anti-ADM antibody fragment or anti-ADM non-Ig scaffold according to the present invention, wherein said anti-ADM antibody or said anti-ADM antibody fragment or anti-ADM non-Ig scaffold is an ADM stabilizing antibody or an ADM stabilizing antibody fragment or an ADM stabilizing non-Ig scaffold that enhances the half-life (t½; half retention time) of adrenomedullin in serum, blood, plasma at least 10%, preferably at least 50%, more preferably >50%, most preferably >100%.

The half-life (half retention time) of ADM may be determined in human serum, blood or plasma in absence and presence of an ADM stabilizing antibody or an ADM stabilizing antibody fragment or an ADM stabilizing non-Ig scaffold, respectively, using an immunoassay for the quantification of ADM.

The following steps may be conducted:
ADM may be diluted in human citrate plasma in absence and presence of an ADM stabilizing antibody or an adrenomedullin stabilizing antibody fragment or an adrenomedullin stabilizing non-Ig scaffold, respectively, and may be incubated at 24° C.
Aliquots are taken at selected time points (e.g. within 24 hours) and degradation of ADM may be stopped in said aliquots by freezing at −20° C.
The quantity of ADM may be determined by a hADM immunoassay directly, if the selected assay is not influenced by the stabilizing antibody. Alternatively, the aliquot may be treated with denaturing agents (like HCl) and, after clearing the sample (e.g. by centrifugation) the pH can be neutralized and the ADM-quantified by an ADM immunoassay. Alternatively, non-immunoassay technologies (e.g. RP-HPLC) can be used for ADM-quantification.
The half-life of ADM is calculated for ADM incubated in absence and presence of an ADM stabilizing antibody or an adrenomedullin stabilizing antibody fragment or an adrenomedullin stabilizing non-Ig scaffold, respectively.
The enhancement of half-life is calculated for the stabilized ADM in comparison to ADM that has been incubated in absence of an ADM stabilizing antibody or an adrenomedullin stabilizing antibody fragment or an adrenomedullin stabilizing non-Ig scaffold.

A two-fold increase of the half-life of ADM is an enhancement of half-life of 100%.

Half-life (half retention time) is defined as the period over which the concentration of a specified chemical or drug takes to fall to half its baseline concentration in the specified fluid or blood.

An assay that may be used for the determination of the half-life (half retention time) of adrenomedullin in serum, blood, plasma is described in Example 3.

In a preferred embodiment said anti-ADM antibody, anti-ADM antibody fragment or anti-ADM non-Ig scaffold is a non-neutralizing antibody, fragment or scaffold. A neutralizing anti-ADM antibody, anti-ADM antibody fragment or anti-ADM non-Ig scaffold would block the bioactivity of ADM to nearly 100%, to at least more than 90%, preferably to at least more than 95%. In other words, this means that said non-neutralizing anti-ADM antibody, anti-ADM antibody fragment or anti-ADM non-Ig scaffold blocks the bioactivity of ADM to less than 100%, preferably less than 95% preferably less than 90%.

In an embodiment wherein said non-neutralizing anti-ADM antibody, anti-ADM antibody fragment or anti-ADM non-Ig scaffold blocks the bioactivity of ADM to less than 95% an anti-ADM antibody, anti-ADM antibody fragment or anti-ADM non-Ig scaffold that would block the bioactivity of ADM to more than 95% would be outside of the scope of said embodiment. This means in one embodiment that the bioactivity is reduced to 95% or less but not more, preferably to 90% or less, more preferably to 80% or less, more preferably to 50% or less but not more.

In one embodiment of the invention the non-neutralizing antibody is an antibody binding to a region of at least 5 amino acids within the sequence of amino acid 1-21 of mature human ADM (SEQ ID No.: 9), or an antibody binding to a region of at least 5 amino acids within the sequence of amino acid 1-19 of mature murine ADM (SEQ ID No.: 12).

In another preferred embodiment of the invention the non-neutralizing antibody is an antibody binding to a region of at least 4 amino acids within the sequence of amino acid 1-21 of mature human ADM (SEQ ID No.: 9), or an antibody binding to a region of at least 5 amino acids within the sequence of amino acid 1-19 of mature murine ADM (SEQ ID No.: 12).

In a specific embodiment according to the present invention a non-neutralizing anti-ADM antibody or anti-ADM antibody fragment or ADM non-Ig scaffold is used, wherein said anti-ADM antibody or an anti-ADM antibody fragment blocks the bioactivity of ADM to less than 80%, preferably less than 50% (of baseline values). It has to be understood that said limited blocking of the bioactivity (meaning reduction of the bioactivity) of ADM occurs even at excess concentration of the antibody, fragment or scaffold, meaning an excess of the antibody, fragment or scaffold in relation to ADM. Said limited blocking is an intrinsic property of the ADM binder itself in said specific embodiment. This means that said antibody, fragment or scaffold has a maximal inhibition of 80% or 50% respectively. In a preferred embodiment said anti-ADM antibody, anti-ADM antibody fragment or anti-ADM non-Ig scaffold would block the bioactivity/reduce the bioactivity of anti-ADM to at least 5%. The stated above means that approximately 20% or 50% or even 95% residual ADM bioactivity remains present, respectively.

Thus, in accordance with the present invention the provided anti-ADM antibodies, anti-ADM antibody fragments, and anti-ADM non-Ig scaffolds do not neutralize the respective ADM bioactivity.

The bioactivity is defined as the effect that a substance takes on a living organism or tissue or organ or functional unit in vivo or in vitro (e.g. in an assay) after its interaction. In case of ADM bioactivity this may be the effect of ADM in a human recombinant ADM receptor cAMP functional assay. Thus, according to the present invention bioactivity is defined via an ADM receptor cAMP functional assay.

The following steps may be performed in order to determine the bioactivity of ADM in such an assay:
Dose response curves are performed with ADM in said human recombinant ADM receptor cAMP functional assay.
The ADM concentration of half-maximal cAMP stimulation may be calculated.
At constant half-maximal cAMP-stimulating ADM concentrations dose response curves (up to 100 μg/ml final concentration) are performed by an ADM stabilizing antibody or ADM stabilizing antibody fragment or ADM stabilizing non-Ig scaffold, respectively.

A maximal inhibition in said ADM bioassay of 50% means that said anti-ADM antibody or said anti-ADM antibody fragment or said anti-ADM non-Ig scaffold, respectively, blocks the bioactivity of ADM to 50% of baseline values. A maximal inhibition in said ADM bioassay of 80% means that said anti-ADM antibody or said anti-adrenomedullin antibody fragment or said anti-adrenomedullin non-Ig scaffold, respectively, blocks the bioactivity of ADM to 80%. This is in the sense of blocking the ADM bioactivity to not more than 80%. This means approximately 20% residual ADM bioactivity remains present.

However, by the present specification and in the above context the expression “blocks the bioactivity of ADM” in relation to the herein disclosed anti-ADM antibodies, anti-ADM antibody fragments, and anti-ADM non-Ig scaffolds should be understood as mere decreasing the bioactivity of ADM from 100% to 20% remaining ADM bioactivity at maximum, preferably decreasing the ADM bioactivity from 100% to 50% remaining ADM bioactivity; but in any case there is ADM bioactivity remaining that can be determined as detailed above.

The bioactivity of ADM may be determined in a human recombinant Adrenomedullin receptor cAMP functional assay (Adrenomedullin Bioassay) according to Example 2.

In a preferred embodiment a modulating anti-ADM antibody or a modulating anti-ADM antibody fragment or a modulating anti-ADM non-Ig scaffold is used in therapy or prevention according to the present invention.

A “modulating” anti-ADM antibody or a modulating anti-ADM antibody fragment or a modulating anti-ADM non-Ig scaffold is an antibody or antibody fragment or non-Ig scaffold that enhances the half-life (t½ half retention time) of adrenomedullin in serum, blood, plasma at least 10%, preferably at least, 50%, more preferably >50%, most preferably >100% and blocks the bioactivity of ADM to less than 80%, preferably less than 50% and said anti-ADM antibody, anti-ADM antibody fragment or anti-ADM non-Ig scaffold would block the bioactivity of ADM to at least 5%. These values related to half-life and blocking of bioactivity have to be understood in relation to the before-mentioned assays in order to determine these values. This is in the sense of blocking the ADM bioactivity of not more than 80% or not more than 50%, respectively.

Such a modulating anti-ADM antibody or modulating anti-ADM antibody fragment or a modulating anti-ADM non-Ig scaffold offers the advantage that the dosing of the administration is facilitated. The combination of partially blocking or partially reducing ADM bioactivity and increase of the in vivo half-life (increasing the ADM bioactivity) leads to beneficial simplicity of anti-ADM antibody or an anti-ADM antibody fragment or anti-ADM non-Ig scaffold dosing. In a situation of excess of endogenous ADM the activity lowering effect is the major impact of the antibody or fragment or scaffold, limiting the (negative) effect of ADM. In case of low or normal endogenous ADM concentrations, the biological effect of anti-ADM antibody or anti-ADM antibody fragment or anti-ADM non-Ig scaffold is a combination of lowering (by partially blocking) and increase by increasing the ADM half-life. Thus, the non-neutralizing and modulating anti-ADM antibody or anti-ADM antibody fragment or anti-ADM non-Ig scaffold acts like an ADM bioactivity buffer in order to keep the bioactivity of ADM within a certain physiological range.

In a specific embodiment of the invention the antibody is a monoclonal antibody or a fragment thereof. In one embodiment of the invention the anti-ADM antibody or the anti-ADM antibody fragment is a human or humanized antibody or derived therefrom. In one specific embodiment one or more (murine) CDR's are grafted into a human antibody or antibody fragment.

Subject matter of the present invention in one aspect is a human or humanized CDR-grafted antibody or antibody fragment thereof that binds to ADM, wherein the human or humanized CDR-grafted antibody or antibody fragment thereof comprises an antibody heavy chain (H chain) comprising:

(SEQ ID No.: 19)
GYTFSRYW,
(SEQ ID No.: 20)
ILPGSGST
and/or
(SEQ ID No.: 21)
TEGYEYDGFDY

and/or further comprises an antibody light chain (L chain) comprising:

(SEQ ID No.: 22)
QSIVYSNGNTY,
RVS (not part of the Sequencing Listing)
and/or
(SEQ ID No.: 23)
FQGSHIPYT.

In one specific embodiment of the invention subject matter of the present invention is a human or humanized monoclonal antibody that binds to ADM or an antibody fragment thereof that binds to ADM wherein the heavy chain comprises at least one CDR selected from the group comprising: GYTFSRYW (SEQ ID No.: 19), ILPGSGST (SEQ ID No.: 20), TEGYEYDGFDY (SEQ ID No.: 21)

(SEQ ID No.: 19)
GYTFSRYW,
(SEQ ID No.: 20)
ILPGSGST
(SEQ ID No.: 21)
TEGYEYDGFDY

and wherein the light chain comprises at least one CDR selected from the group comprising:

(SEQ ID No.: 22)
QSIVYSNGNTY,
RVS (not part of the Sequencing Listing)
(SEQ ID No.: 23)
FQGSHIPYT.

In a more specific embodiment of the invention subject matter of the invention is a human monoclonal antibody that binds to ADM or an antibody fragment thereof that binds to ADM wherein the heavy chain comprises the sequences:

(SEQ ID No.: 19)
GYTFSRYW,
(SEQ ID No.: 20)
ILPGSGST
(SEQ ID No.: 21)
TEGYEYDGFDY

and wherein the light chain comprises the sequences:

(SEQ ID No.: 22)
QSIVYSNGNTY,
RVS (not part of the Sequencing Listing)
(SEQ ID No.: 23)
FQGSHIPYT.

In a very specific embodiment, the anti-ADM antibody comprises a sequence selected from the group comprising: SEQ ID No. 24, 25, 26, 27, 28, 29, 30, 31, 32 and 33.

The anti-ADM antibody or anti-ADM antibody fragment or anti-ADM non-Ig scaffold according to the present invention exhibits an affinity towards human ADM in such that affinity constant is greater than 10⁻⁷ M, preferred 10⁻⁸ M, preferred affinity is greater than 10⁻⁹ M, most preferred higher than 10⁻¹⁰ M. A person skilled in the art knows that it may be considered to compensate lower affinity by applying a higher dose of compounds and this measure would not lead out-of-the-scope of the invention. The affinity constants may be determined according to the method as described in Example 1.

In more particular embodiments of the present invention, said antibody or fragment comprises a sequence selected from the group comprising:

(AM-VH-C)
SEQ ID No.: 24
QVQLQQSGAELMKPGASVKISCKATGYTFSRYWIEWVKQRPGHGLEWIGEILPGSGSTNYNE
KFKGKATITADTSSNTAYMQLSSLTSEDSAVYYCTEGYEYDGFDYWGQGTTLTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKRVEPK
(AM-VH1)
SEQ ID No.: 25
QVQLVQSGAEVKKPGSSVKVSCKASGYTFSRYWISWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH2-E40)
SEQ ID No.: 26
QVQLVQSGAEVKKPGSSVKVSCKASGYTFSRYWIEWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH3-T26-E55)
SEQ ID No.: 27
QVQLVQSGAEVKKPGSSVKVSCKATGYTFSRYWISWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH4-T26-E40-E55)
SEQ ID No.: 28
QVQLVQSGAEVKKPGSSVKVSCKATGYTFSRYWIEWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VL-C)
SEQ ID No.: 29
DVLLSQTPLSLPVSLGDQATISCRSSQSIVYSNGNTYLEWYLQKPGQSPKLLIYRVSNRFSGVP
DRFSGSGSGTDFTLKISRVEAEDLGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
(AM-VL1)
SEQ ID No.: 30
DVVMTQSPLSLPVTLGQPASISCRSSQSIVYSNGNTYLNWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
(AM-VL2-E40)
SEQ ID No.: 31
DVVMTQSPLSLPVTLGQPASISCRSSQSIVYSNGNTYLEWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC.

In even more particular embodiments of the present invention, said antibody or fragment comprises the following sequence as a heavy chain:

SEQ ID No.: 32
QVQLVQSGAEVKKPGSSVKVSCKASGYTFSRYWIEWVRQAPGQGLEWIGEILPGSGSTNYNQ
KFQGRVTITADTSTSTAYMELSSLRSEDTAVYYCTEGYEYDGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFScSVMHEALHNHY
TQKSLSLSPGK

and comprises the following sequence as a light chain:

SEQ ID No.: 33
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC.

In a more specific embodiment of the invention the antibody comprises the following sequence as a heavy chain:

SEQ ID No.: 32
QVQLVQSGAEVKKPGSSVKVSCKASGYTFSRYWIEWVRQAPGQGLEWIGEILPGSGSTNYNQ
KFQGRVTITADTSTSTAYMELSSLRSEDTAVYYCTEGYEYDGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYP
SDIAEWESNGQPENNYKTTPPVLDSDGSFFLYSKLTVDKSRWQQGNVFSCSVMHEALHNH
YTQKSLSLSPGK

or a sequence that is >95% identical to it, preferably >98%, preferably >99% and comprises the following sequence as a light chain:

SEQ ID No.: 33
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC

or a sequence that is >95% identical to it, preferably >98%, preferably >99%
wherein the heavy chain comprises the sequences:

CDR1:
SEQ ID No.: 19
GYTFSRYW
CDR2:
SEQ ID No.: 20
ILPGSGST
CDR3:
SEQ ID No.: 21
TEGYEYDGFDY

and wherein the light chain comprises the sequences:

CDR1:
SEQ ID No.: 22
QSIVYSNGNTY
CDR2:
RVS
CDR3:
SEQ ID No.: 23
FQGSHIPYT.

This means, in one embodiment of the invention the CDR's do not exhibit any variations of the sequence. Any variation of the above sequence is outside of the CDR's in said embodiment.

To assess the identity between two amino acid sequences, a pairwise alignment is performed. Identity defines the percentage of amino acids with a direct match in the alignment.

In more particular embodiments of the present invention, said antibody or fragment thereof comprises a heavy chain Fab fragment comprising SEQ ID NO: 35.

In more particular embodiments of the present invention, said antibody or fragment thereof comprises a light chain Fab fragment comprising SEQ ID NO: 33.

In more particular embodiments of the present invention, said antibody or fragment thereof comprises a heavy chain Fab fragment comprising SEQ ID NO: 35 and comprises a light chain Fab fragment comprising SEQ ID NO: 33.

In more particular embodiments of the present invention, said antibody or fragment thereof comprises a heavy chain variable region portion from SEQ ID NO: 35.

In more particular embodiments of the present invention, said antibody or fragment thereof comprises a light chain variable region portion from SEQ ID NO: 33.

In more particular embodiments of the present invention, said antibody or fragment thereof comprises a heavy chain variable region portion from SEQ ID NO: 35 and comprises a light chain variable region portion from SEQ ID NO: 33.

In more particular embodiments of the present invention, the fragment is a ScFv and wherein the ScFv comprises a heavy chain variable region portion from SEQ ID NO: 35 and comprises a light chain variable region portion from SEQ ID NO: 33, and wherein the heavy chain variable region portion from SEQ ID NO: 35 and a light chain variable region portion from SEQ ID NO: 33 are linked together via linker.

In more particular embodiments of the present invention, said antibody or fragment thereof is a fragment, wherein the fragment is a Fab fragment and wherein the Fab fragment comprises a heavy chain Fab fragment comprising SEQ ID NO: 35 and comprises a light chain Fab fragment comprising SEQ ID NO: 33.

In embodiments of the present invention, the anti-ADM antibody or anti-ADM antibody fragment for is administered in a dose of at least 0.5 mg/kg body weight, particularly at least 1.0 mg/kg body weight, more particularly, from 1.0 to 20.0 mg/kg body weight, e.g., from 2.0 to 10 mg/kg body weight, from 2.0 to 8.0 mg/kg body weight, or from 2.0 to 5.0 mg/kg body weight.

With the above context, the following consecutively numbered embodiments provide further specific aspects of the invention:
1. Anti-ADM antibody or anti-ADM antibody fragment or anti-ADM non-Ig scaffold for use in therapy or prevention of side effects of immune effector cell administration to a cancer patient in the frame of an immune effector cell therapy (IECT), wherein said anti-ADM antibody or anti-ADM antibody fragment or anti-ADM non-Ig scaffold is administered prior to or after administration of said immune effector cells to said patient.
2. Anti-ADM antibody or anti-ADM antibody fragment or anti-ADM non-Ig scaffold for use according to item 1, wherein said side effects of immune effector cell administration in the frame of an immune effector cell therapy (IECT) are associated with endothelial dysfunction.
3. Anti-ADM antibody or anti-ADM antibody fragment or anti-ADM non-Ig scaffold for use according to item 1 or 2, wherein said anti-ADM antibody or anti-ADM antibody fragment or anti-ADM non-Ig scaffold is used for reducing immune effector cell therapy side effects.
4. Anti-ADM antibody or anti-ADM antibody fragment or anti-ADM non-Ig scaffold for use according to items 1 to 3, wherein said immune effector cell therapy side effects are selected from the group comprising cytokine release syndrome (CRS), and immune effector cell-associated neurotoxicity syndrome (ICANS).
5. Anti-ADM antibody or anti-ADM antibody fragment or anti-ADM non-Ig scaffold for use according to items 1 to 4, wherein said immune effector cell therapy (IECT) is selected from the group comprising Chimeric Antigen Receptor T-cell (CAR-T) therapy, natural killer cell (NK) therapy, chimeric antigen receptor natural killer cell (CAR-NK) therapy, T cell receptor-engineered T cell (TCR T) therapy, tumor-infiltrating T cell (TIT)), and cytokine-induced killer cell (CIK) therapy.
6. Anti-ADM antibody or anti-ADM antibody fragment or anti-ADM non-Ig scaffold for use according to items 1 to 5, wherein said therapy comprises the administration of further medication for the treatment of immune effector cell therapy side effects.
7. Anti-ADM antibody or anti-ADM antibody fragment or anti-ADM non-Ig scaffold for use according to item 6, wherein said further medication is selected from the group comprising antihistamins, non-steroidal anti-inflammatory drugs (NSAIDs), immunosuppressive drugs, vasopressors, fluids and oxygen.
8. Anti-ADM antibody or anti-ADM antibody fragment or anti-ADM non-Ig scaffold for use according to item 7, wherein said immunosuppressive drugs are one or more drugs selected from the group comprising anti-IL-6 receptor antibody (e.g. tocilizumab), anti-IL-6 antibody (e.g. siltuximab), anti-TNF antibody (e.g. etanercept, infliximab), anti-IL-1 antibody (e.g. anakinra) and corticosteroids.
9. Anti-ADM antibody or anti-ADM antibody fragment or anti-ADM non-Ig scaffold for use according to item 8, wherein said corticosteroid is selected from the group comprising cortisone, hydrocortisone, fludrocortisone, dexamethasone, betamethasone, prednisolone, methylprednisolone, and prednisone.
10. Anti-ADM antibody or anti-ADM antibody fragment or anti-ADM non-Ig scaffold for use according to items 1 to 9, wherein said patient is having a level of Pro-Adrenomedullin or fragments thereof in a sample of bodily fluid above a threshold.
11. Anti-ADM antibody or anti-ADM antibody fragment or anti-ADM non-Ig scaffold for use according to item 10, wherein said Pro-Adrenomedullin or fragment thereof is selected from the group comprising Pro-Adrenomedullin (SEQ ID No.: 2), mature PAMP (SEQ ID No.: 4), PAMP-Gly (SEQ ID No.: 3), MR-proADM (SEQ ID No.: 5), ADM-Gly (SEQ ID No.: 6), mature ADM (SEQ ID No.: 7) and CT-proADM (SEQ ID No.: 8).
12. Anti-ADM antibody or anti-ADM antibody fragment or anti-ADM non-Ig scaffold for use according to item 11, wherein said fragment of Pro-Adrenomedullin is MR-proADM and the threshold of the level of MR-proADM in a sample of bodily fluid of said patient is between 0.5 and 2 nmol/L, more particular between 0.6 and 1.5 nmol/L, even more particular between 0.7 and 1 nmol/L, most particular said threshold is 0.8 nmol/L
or is an x-fold of the median level of MR-proADM in a healthy population, in particular in the range between 1.2-fold and 4.9-fold, more particular between 1.5-fold and 3.7-fold, even more particular between 1.7-fold and 2.4-fold, most particular said threshold is 2.0-fold of the median of the level of MR-proADM in a healthy population.
13. Anti-ADM antibody or anti-ADM antibody fragment or anti-ADM non-Ig scaffold for use according to item 11, wherein said fragment of Pro-Adrenomedullin is mature PAMP and the threshold of the level of mature PAMP in a sample of bodily fluid of said patient is between 0.7 and 1.2 pmol/L, more particular between 0.8 and 1.0 pmol/L, most particular said threshold is 0.9 pmol/L,
or is an x-fold of the mean level of mature PAMP in a healthy population, in particular in the range between the 1.4-fold and 2.4-fold, more particular in the range between 1.6-fold and 2.0-fold, most particular said threshold is the 1.8-fold of the mean of the level of mature PAMP in a healthy population.
14. Anti-ADM antibody or anti-ADM antibody fragment or anti-ADM non-Ig scaffold for use according to item 11, wherein said fragment of Pro-Adrenomedullin is PAMP-Gly and the threshold of the level of PAMP-Gly in a sample of bodily fluid of said patient is between 1.5 and 2.4 pmol/L, even more preferred between 1.8 and 2.2 pmol/L, most preferred said threshold is 2.0 pmol/L,
or is an x-fold of the mean level of PAMP-Gly in a healthy population, in particular in the range between the 1.3-fold and 2.1-fold, more particular in the range between 1.6-fold and 1.9-fold, most particular said threshold is the 1.7-fold of the mean of the level of PAMP-Gly in a healthy population.
15. Anti-ADM antibody or anti-ADM antibody fragment or anti-ADM non-Ig scaffold for use according to item 11, wherein said fragment of Pro-Adrenomedullin is ADM-Gly and the threshold of the level of ADM-Gly in a sample of bodily fluid of said patient is between 25 and 125 μg/ml, more preferred between 30 and 100 μg/ml, even more particular between 35 and 75 μg/ml, most particular said threshold is 40 μg/ml,
or is an x-fold of the median level of ADM-Gly in a healthy population, in particular in the range between the 0.9-fold and 4.6-fold, more particular in the range between the 1.1-fold and 3.7-fold, more particular in the range between 1.3-fold and 2.8-fold, most particular said threshold is the 1.5-fold of the median of the level of ADM-Gly in a healthy population.
16. Anti-ADM antibody or anti-ADM antibody fragment or anti-ADM non-Ig scaffold for use according to item 11, wherein said fragment of Pro-Adrenomedullin is mature ADM and the threshold of the level of mature ADM in a sample of bodily fluid of said patient is between 35 and 125 μg/ml, more particular between 40 and 100 μg/ml, even more particular between 50 and 90 μg/ml, most particular said threshold is 70 μg/ml,
or is an x-fold of the median level of mature ADM in a healthy population, in particular in the range between the 2.9-fold and 7.3-fold, more particular in the range between 3.6-fold and 6.6-fold, most particular said threshold is the 5.1-fold of the median of the level of mature ADM in a healthy population.
17. Anti-ADM antibody or anti-ADM antibody fragment or anti-ADM non-Ig scaffold for use according to item 11, wherein said fragment of Pro-Adrenomedullin is CT-proADM and the threshold of the level of CT-proADM in a sample of bodily fluid of said patient is between 75 and 350 pmol/L, more particular between 100 and 250 pmol/L, even more particular between 125 and 200 pmol/L, most particular said threshold is 150 pmol/L,
or is an x-fold of the median level of CT-proADM in a healthy population, in particular in the range between the 1.0-fold and 4.5-fold, more particular in the range between the 1.3-fold and 3.2-fold, more particular in the range between 1.6-fold and 2.6-fold, most particular said threshold is the 1.9-fold of the median of the level of CT-proADM in a healthy population.
18. Anti-ADM antibody or anti-ADM antibody fragment or anti-ADM non-Ig scaffold for use according to cany of items 10 to 17, wherein said sample is a bodily fluid sample selected from the group comprising whole blood, plasma or serum.
19. Anti-ADM antibody or anti-ADM antibody fragment or anti-ADM non-Ig scaffold for according to items 1 to 18, wherein said anti-ADM antibody or anti-ADM fragment or anti-ADM non-Ig scaffold binds to the N-terminal part (amino acids 1-21) of ADM:

(SEQ ID No.: 9)
YRQSMNNFQGLRSFGCRFGTC.

20. Anti-ADM antibody or anti-ADM antibody fragment or anti-ADM non-Ig scaffold for use according to items 1 to 19, wherein said anti-ADM antibody or anti-ADM antibody fragment or anti-ADM non-Ig scaffold recognizes and binds to the N-terminal moiety (comprising amino acid 1) of ADM.
21. Anti-ADM antibody or anti-ADM antibody fragment or anti-ADM non-Ig scaffold for use according to items 1 to 20, wherein said antibody or fragment is a monoclonal antibody or fragment that binds to ADM, wherein the heavy chain comprises the sequences:

CDR1 (SEQ ID No.: 19):
GYTFSRYW
CDR2 (SEQ ID No.: 20):
ILPGSGST
CDR3 (SEQ ID No.: 21):
TEGYEYDGFDY

and wherein the light chain comprises the sequences:

CDR1 (SEQ ID No.: 22):
QSIVYSNGNTY
CDR2:
RVS
CDR3 (SEQ ID No.: 23):
FQGSHIPYT.

22. Anti-ADM antibody or anti-ADM antibody fragment or anti-ADM non-Ig scaffold for use according to items 1 to 21, wherein said anti-ADM antibody or anti-ADM antibody fragment comprises a sequence selected from the group comprising the following sequences as a VH region:

(AM-VH-C)
SEQ ID No.: 24
QVQLQQSGAELMKPGASVKISCKATGYTFSRYWIEWVKQRPGHGLEWIGEILPGSGSTN
YNEKFKGKATITADTSSNTAYMQLSSLTSEDSAVYYCTEGYEYDGFDYWGQGTTLTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPK
(AM-VH1)
SEQ ID No.: 25
QVQLVQSGAEVKKPGSSVKVSCKASGYTFSRYWISWVRQAPGQGLEWMGRILPGSGST
NYAQKFQGRVTITADESTSTAYMELSSLRSEDTAVYYCTEGYEYDGFDYWGQGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK
(AM-VH2-E40)
SEQ ID No.: 26
QVQLVQSGAEVKKPGSSVKVSCKASGYTFSRYWIEWVRQAPGQGLEWMGRILPGSGST
NYAQKFQGRVTITADESTSTAYMELSSLRSEDTAVYYCTEGYEYDGFDYWGQGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK
(AM-VH3-T26-E55)
SEQ ID No.: 27
QVQLVQSGAEVKKPGSSVKVSCKATGYTFSRYWISWVRQAPGQGLEWMGEILPGSGST
NYAQKFQGRVTITADESTSTAYMELSSLRSEDTAVYYCTEGYEYDGFDYWGQGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK
(AM-VH4-T26-E40-E55)
SEQ ID No.: 28
QVQLVQSGAEVKKPGSSVKVSCKATGYTFSRYWIEWVRQAPGQGLEWMGEILPGSGST
NYAQKFQGRVTITADESTSTAYMELSSLRSEDTAVYYCTEGYEYDGFDYWGQGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK

or a sequence that is >95% identical to it, and comprises a sequence selected from the group comprising the following sequences as a VL region:

SEQ ID No.: 29 (AM-VL-C):
DVLLSQTPLSLPVSLGDQATISCRSSQSIVYSNGNTYLEWYLQKPGQSPKLLIYRVSNRFS
GVPDRFSGSGSGTDFTLKISRVEAEDLGVYYCFQGSHIPYTFGGGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
(AM-VL1)
SEQ ID No.: 30
DVVMTQSPLSLPVTLGQPASISCRSSQSIVYSNGNTYLNWFQQRPGQSPRRLIYRVSNRDS
GVPDRFSGSGSGTDFTLKISRVEAEDVGVYYCFQGSHIPYTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
(AM-VL2-E40)
SEQ ID No.: 31
DVVMTQSPLSLPVTLGQPASISCRSSQSIVYSNGNTYLEWFQQRPGQSPRRLIYRVSNRDS
GVPDRFSGSGSGTDFTLKISRVEAEDVGVYYCFQGSHIPYTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

or a sequence that is >95% identical to it.
23. Anti-ADM antibody or anti-ADM antibody fragment or anti-ADM non-Ig scaffold for use according to items 1 to 22, wherein said anti-ADM antibody or anti-ADM antibody fragment comprises the following sequence as a heavy chain SEQ ID No.: 32

SEQ ID No.: 32
QVQLVQSGAEVKKPGSSVKVSCKASGYTFSRYWIEWVRQAPGQGLEWIGEILPGSGSTN
YNQKFQGRVTITADTSTSTAYMELSSLRSEDTAVYYCTEGYEYDGFDYWGQGTTVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKS
RWQQGNVFScSVMHEALHNHYTQKSLSLSPGK

or a sequence that is >95% identical to it,
and comprises the following sequence as a light chain SEQ ID No.: 33

DVVLTQSPLSLPVTLGQPASISCRSSQSIVYSNGNTYLEWYLQRPGQSPRLLIYRVSNRFS
GVPDRFSGSGSGTDFTLKISRVEAEDVGVYYCFQGSHIPYTFGGGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

or a sequence that is >95% identical to it.
24. Anti-ADM antibody or anti-ADM antibody fragment or anti-ADM non-Ig scaffold for use according to items 1 to 21 or 23, wherein said antibody or fragment thereof comprises a heavy chain Fab Fragment comprising SEQ ID NO: 35.
25. Anti-ADM antibody or anti-ADM antibody fragment or anti-ADM non-Ig scaffold for use according to items 1 to 21, 23 or 24, wherein said antibody or fragment thereof comprises a light chain Fab Fragment comprising SEQ ID NO: 33.
26. Anti-ADM antibody or anti-ADM antibody fragment or anti-ADM non-Ig scaffold for use according to items 1 to 21 or 23 to 25, wherein said antibody or fragment thereof comprises a heavy chain Fab Fragment comprising SEQ ID NO: 35 and comprises a light chain Fab Fragment comprising SEQ ID NO: 33.
27. Anti-ADM antibody or anti-ADM antibody fragment or anti-ADM non-Ig scaffold for use according to items 1 to 21 or 23 to 26, wherein said antibody or fragment thereof comprises a heavy chain variable region portion of SEQ ID NO: 35.
28. Anti-ADM antibody or anti-ADM antibody fragment or anti-ADM non-Ig scaffold for use according to items 1 to 21 or 23 to 27, wherein said antibody or fragment thereof comprises a light chain variable region portion of SEQ ID NO: 33.
29. Anti-ADM antibody or anti-ADM antibody fragment or anti-ADM non-Ig scaffold for use according to items 1 to 21 or 23 to 28, wherein said antibody or fragment thereof comprises a heavy chain variable region portion of SEQ ID NO: 35 and comprises a light chain variable region portion of SEQ ID NO: 33.
30. Anti-ADM antibody or anti-ADM antibody fragment or anti-ADM non-Ig scaffold for use according to items 1 to 21 or 23 to 29, wherein said antibody or fragment thereof is a fragment, wherein the fragment is a ScFv and wherein the ScFv comprises a heavy chain variable region portion of SEQ ID NO: 35 and comprises a light chain variable region portion of SEQ ID NO: 33 and wherein the ScFv further comprises a linker connecting the heavy chain variable region portion of SEQ ID NO: 35 and the light chain variable region portion of SEQ ID NO: 33.
31. Anti-ADM antibody or anti-ADM antibody fragment or anti-ADM non-Ig scaffold for use according to items 1 to 21 or 23 to 30, wherein said antibody or fragment thereof comprises a heavy chain Fab region comprising SEQ ID NO: 35 and comprises a light chain variable region comprising SEQ ID NO: 33.
32. Anti-ADM antibody or anti-ADM antibody fragment or anti-ADM non-Ig scaffold for use according to items 1 to 21 or 23 to 31, wherein said antibody or fragment thereof is a fragment, wherein the fragment is a ScFv and wherein the ScFv comprises a heavy chain variable region comprising SEQ ID NO: 35 and comprises a light chain variable region comprising SEQ ID NO: 33.
33. Anti-ADM antibody or anti-ADM antibody fragment or anti-ADM non-Ig scaffold for use according to items 1 to 21 or 23 to 32, wherein said antibody or fragment thereof is a fragment, wherein the fragment is a Fab fragment and wherein the Fab fragment comprises a heavy chain Fab region comprising SEQ ID NO: 35 and comprises a light chain variable region comprising SEQ ID NO: 33.

### FIGURE DESCRIPTION

FIG. 1A: Illustration of antibody formats—Fv and scFv-Variants.

FIG. 1B: Illustration of antibody formats—heterologous fusions and bifunctional antibodies.

FIG. 1C: Illustration of antibody formats—bivalental antibodies and bispecific antibodies.

FIG. 2A: Dose response curve of human ADM. Maximal cAMP stimulation was adjusted to 100% activation.

FIG. 2B: Dose/inhibition curve of human ADM 22-52 (ADM-receptor antagonist) in the presence of 5.63 nM hADM.

FIG. 2C: Dose/inhibition curve of CT-H in the presence of 5.63 nM hADM.

FIG. 2D: Dose/inhibition curve of MR-H in the presence of 5.63 nM hADM.

FIG. 2E: Dose/inhibition curve of NT-H in the presence of 5.63 nM hADM.

FIG. 2F: Dose response curve of mouse ADM. Maximal cAMP stimulation was adjusted to 100% activation.

FIG. 2G: Dose/inhibition curve of human ADM 22-52 (ADM-receptor antagonist) in the presence of 0.67 nM mADM.

FIG. 2H: Dose/inhibition curve of CT-M in the presence of 0.67 nM mADM.

FIG. 2I: Dose/inhibition curve of MR-M in the presence of 0.67 nM mADM.

FIG. 2J: Dose/inhibition curve of NT-M in the presence of 0.67 nM mADM.

FIG. 2K: Shows the inhibition of ADM by F(ab)₂ NT-M and by Fab NT-M.

FIG. 2L: shows the inhibition of ADM by F(ab)₂ NT-M and by Fab NT-M.

FIG. 3: This figure shows a typical hADM dose/signal curve. And an hADM dose signal curve in the presence of 100 μg/mL antibody NT-H.

FIG. 4: This figure shows the stability of hADM in human plasma (citrate) in absence and in the presence of NT-H antibody.

FIG. 5: Alignment of the Fab with homologous human framework sequences.

FIG. 6: ADM-concentration in healthy human subjects after NT-H antibody application at different doses up to 60 days.

FIG. 7: Representative calibration curve of the ADM-Gly Assay with synthetic human 1-53 ADM-Gly

FIG. 8: Frequency distribution (histogram) of ADM-Gly (pg/mL) in self-reported healthy individuals (n=128)

FIGS. 9A-9B: Concentrations of bio-ADM—(FIG. 9A) and ADM-Gly (FIG. 9B) in cancer patients prior to start of CAR T-cell administration in the frame of a chimeric antigen receptor T-cell therapy (CAR T-cell) dependent on the Cytokine Release Syndrome (CRS) Grading <2 or ≥2 following the CAR T-cell therapy. Shown are box and whiskers plots (with median, 25th and 75th percentiles and min and max data indicated).

FIGS. 10A-10B: Time course of bio-ADM (FIG. 10A) and ADM-Gly (FIG. 10B) after start of CAR T-cell administration in the frame of CAR T-cell therapy dependent on the Cytokine Release Syndrome (CRS) Grading <2 or >2 following the CAR T-cell therapy. As blood draws were not taken at the exact same time points after start of CAR T-cell therapy for all patients, bio-ADM- and ADM-Gly measurements were grouped in time intervals of four days, e.g. >0 and <4 days, >4 and <8 days, >8 and <12 days, >12 and <16 days, >16 days, after start of CAR T-cell therapy. Displayed are bars for median with interquartile range

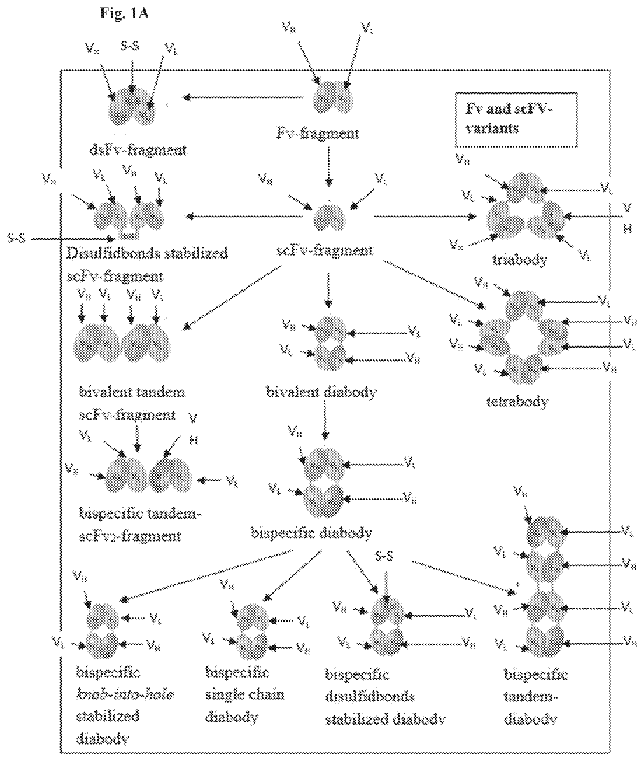

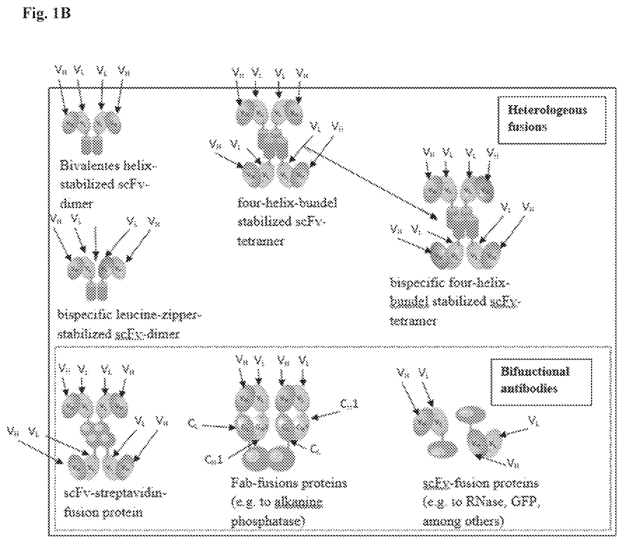

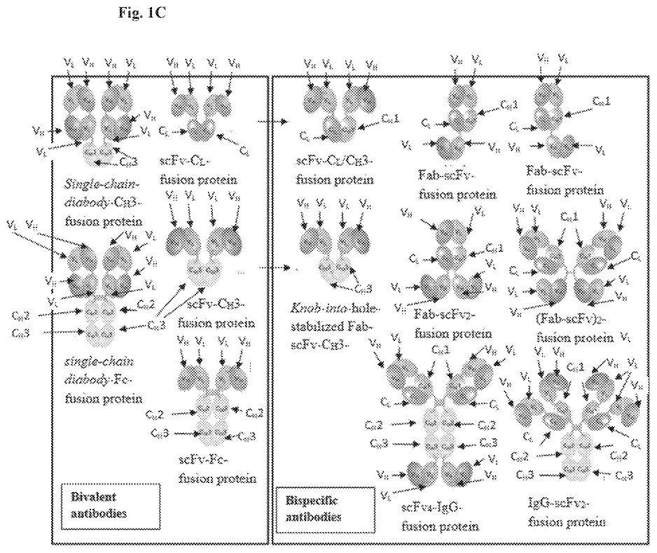

### REFERENCES

Ayala Ceja, M., Khericha, M., Harris, C. M., Puig-Saus, C., & Chen, Y. Y. (2024). CAR-T cell manufacturing: major process parameters and next-generation strategies. Journal of Experimental Medicine, 221(2), e20230903.
Bálint, L., Nelson-Maney, N. P., Tian, Y., Serafin, S. D., & Caron, K. M. (2023). Clinical Potential of Adrenomedullin Signaling in the Cardiovascular System. Circulation Research, 132(9), 1185-1202. https://doi.org/10.1161/CIRCRESAHA.123.321673
Chistiakov, D. A., Orekhov, A. N., & Bobryshev, Y. V. (2015). Endothelial barrier and its abnormalities in cardiovascular disease. Frontiers in Physiology, 6, 158540.
Chohan, K. L., Siegler, E. L., & Kenderian, S. S. (2023). CAR-T cell therapy: the efficacy and toxicity balance. Current Hematologic Malignancy Reports, 18(2), 9-18.
Claesson-Welsh, L. (2015). Vascular permeability—the essentials. Upsala Journal of Medical Sciences, 120(3), 135-143.
Claesson-Welsh, L., Dejana, E., & McDonald, D. M. (2021). Permeability of the endothelial barrier: identifying and reconciling controversies. Trends in Molecular Medicine, 27(4), 314-331.
Eto, T. (2001). A review of the biological properties and clinical implications of adrenomedullin and proadrenomedullin N-terminal 20 peptide (PAMP), hypotensive and vasodilating peptides. Peptides, 22(11), 1693-1711. https://doi.org/https://doi.org/10.1016/S0196-9781(01)₀₀₅₁₃-7
Garcia, M. A., Martin-Santamaria, S., de Pascual-Teresa, B., Ramos, A., Julián, M., & Martinez, A. (2006). Adrenomedullin: a new and promising target for drug discovery. Expert Opinion on Therapeutic Targets, 10(2), 303-317.
Gavriilaki, E., Sakellari, I., Gavriilaki, M., & Anagnostopoulos, A. (2020). A new era in endothelial injury syndromes: toxicity of CAR-T cells and the role of immunity. International Journal of Molecular Sciences, 21(11), 3886.
Gül Wer, E., Balat, A., Cekmen, M., Kiling, M., Sivasli, E., Yürekli, M., & Duman, C. (2006). The effects of anticancer drugs on levels of nitric oxide and adrenomedullin. The Turkish Journal of Pediatrics, 48(3), 202-208.
Lee, D. W., Santomasso, B. D., Locke, F. L., Ghobadi, A., Turtle, C. J., Brudno, J. N., Maus, M. V, Park, J. H., Mead, E., & Pavletic, S. (2019). ASTCT consensus grading for cytokine release syndrome and neurologic toxicity associated with immune effector cells. Biology of Blood and Marrow Transplantation, 25(4), 625-638.
Lee, W. L., & Slutsky, A. S. (2010). Sepsis and endothelial permeability. New England Journal of Medicine, 363(7), 689-691.
Link, W. (2019). Cancer Therapy BT—Principles of Cancer Treatment and Anticancer Drug Development (W. Link (ed.); pp. 7-76). Springer International Publishing. https://doi.org/10.1007/978-3-030-18722-4_2
Neelapu, S. S., Tummala, S., Kebriaei, P., Wierda, W., Gutierrez, C., Locke, F. L., Komanduri, K. V, Lin, Y., Jain, N., & Daver, N. (2018). Chimeric antigen receptor T-cell therapy-assessment and management of toxicities. Nature Reviews Clinical Oncology, 15(1), 47-62.
O Karpinich, N., L Hoopes, S., O Kechele, D., M Lenhart, P., & M Caron, K. (2011). Adrenomedullin function in vascular endothelial cells: insights from genetic mouse models. Current Hypertension Reviews, 7(4), 228-239.
Opal, S. M., & Van Der Poll, T. (2015). Endothelial barrier dysfunction in septic shock. Journal of Internal Medicine, 277(3), 277-293.
Rodrigues, S. F., & Granger, D. N. (2015). Blood cells and endothelial barrier function. Tissue Barriers, 3(1-2), e978720.
Rosen, R. S., Yang, J. H., Pena, J. S., Schloss, R., & Yarmush, M. L. (2023). An in vitro model of the macrophage-endothelial interface to characterize CAR T-cell induced cytokine storm. Scientific Reports, 13(1), 18835.
Soussi, S., dos Santos, C., Jentzer, J. C., Mebazaa, A., Gayat, E., Pöss, J., Schaubroeck, H., Billia, F., Marshall, J. C., & Lawler, P. R. (2023). Distinct host-response signatures in circulatory shock: a narrative review. Intensive Care Medicine Experimental, 11(1). https://doi.org/10.1186/s40635-023-00531-5
Sumransub, N., El Jurdi, N., Chiraphapphaiboon, W., & Maakaron, J. E. (2022). Putting function back in dysfunction: Endothelial diseases and current therapies in hematopoietic stem cell transplantation and cellular therapies. Blood Reviews, 51, 100883.
Sun, D., Shi, X., Li, S., Wang, X., Yang, X., & Wan, M. (2024). CAR-T cell therapy: A breakthrough in traditional cancer treatment strategies. Molecular Medicine Reports, 29(3), 1-9.
van Lier, D., Kox, M., & Pickkers, P. (2020). Promotion of vascular integrity in sepsis through modulation of bioactive adrenomedullin and dipeptidyl peptidase 3. Journal of Internal Medicine, 1-15. https://doi.org/10.1111/joim.13220

### EXAMPLES

### Example 1—Generation of Anti-ADM Antibodies and Determination of their Affinity Constants

Several human and murine antibodies were produced and their affinity constants were determined (see tables 1 and 2). It should be emphasized that the antibodies, antibody fragments and non-Ig scaffolds of the example portion in accordance with the invention are binding to ADM, and thus should be considered as anti-ADM antibodies/antibody fragments/non-Ig scaffolds.

### Peptides/Conjugates for Immunization:

Peptides for immunization were synthesized, see Table 1, (JPT Technologies, Berlin, Germany) with an additional N-terminal Cystein (if no Cystein is present within the selected ADM-sequence) residue for conjugation of the peptides to Bovine Serum Albumin (BSA). The peptides were covalently linked to BSA by using Sulfolink-coupling gel (Perbio-science, Bonn, Germany). The coupling procedure was performed according to the manual of Perbio.

### Mouse Monoclonal Antibody Production:

A Balb/c mouse was immunized with 100 μg Peptide-BSA-Conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intra-venous injection. Splenocytes from the immunized mouse and cells of the myeloma cell line SP2/0 were fused with 1 ml 50% polyethylene glycol for 30s at 37° C. After washing, the cells were seeded in 96-well cell culture plates. Hybrid clones were selected by growing in HAT medium [RPMI 1640 culture medium supplemented with 20% fetal calf serum and HAT-Supplement]. After two weeks the HAT medium is replaced with HT Medium for three passages followed by returning to the normal cell culture medium. The cell culture supernatants were primary screened for antigen specific IgG antibodies three weeks after fusion. The positive tested microcultures were transferred into 24-well plates for propagation. After retesting, the selected cultures were cloned and re-cloned using the limiting-dilution technique and the isotypes were determined (see also Lane, R. D. 1985. J. Immunol. Meth. 81: 223-228; Ziegler et al. 1996. Horm. Metab. Res. 28: 11-15).

Antibodies were produced via standard antibody production methods (Marx et al, 1997. Monoclonal Antibody Production, ATLA 25, 121) and purified via Protein A. The antibody purities were >95% based on SDS gel electrophoresis analysis.

Human Antibodies:

Human Antibodies were produced by means of phage display according to the following procedure: The human naive antibody gene libraries HAL7/8 were used for the isolation of recombinant single chain F-Variable domains (scFv) against adrenomedullin peptide. The antibody gene libraries were screened with a panning strategy comprising the use of peptides containing a biotin tag linked via two different spacers to the adrenomedullin peptide sequence. A mix of panning rounds using non-specifically bound antigen and streptavidin bound antigen were used to minimize background of non-specific binders. The eluted phages from the third round of panning have been used for the generation of monoclonal scFv expressing E. coli strains. Supernatant from the cultivation of these clonal strains has been directly used for an antigen ELISA testing (see also Hust et al. 2011. Journal of Biotechnology 152, 159-170; Schutte et al. 2009. PLoS One 4, e6625). Positive clones have been selected based on positive ELISA signal for antigen and negative for streptavidin coated micro titer plates. For further characterizations the scFv open reading frame has been cloned into the expression plasmid pOPE107 (Hust et al., J. Biotechn. 2011), captured from the culture supernatant via immobilized metal ion affinity chromatography and purified by a size exclusion chromatography.

Affinity Constants: To determine the affinity of the antibodies to ADM, the kinetics of binding of ADM to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare). (Lorenz et al. 2011. Antimicrob Agents Chemother. 55(1): 165-173).

The monoclonal antibodies were raised against the below depicted ADM regions of human and murine ADM, respectively. The following table represents a selection of obtained antibodies used in further experiments. Selection was based on target region:

TABLE 1
ADM immunization peptides

| | | | | Affinity |
|---|---|---|---|---|
| Sequence | | ADM | | constants |
| Number | Antigen/Immunogen | Region | Designation | Kd (M) |
| SEQ ID No.: 9 | YRQSMNNFQGLRSFGCRFGTC | 1-21 | NT-H | 5.9 × 10⁻⁹ |
| SEQ ID No.: 10 | CTVQKLAHQIYQ | 21-32 | MR-H | 2 × 10⁻⁹ |
| SEQ ID No.: 11 | CAPRSKISPQGY-NH₂ | C-42-52 | CT-H | 1.1 × 10⁻⁹ |
| SEQ ID No.: 12 | YRQSMNQGSRSNGCRFGTC | 1-19 | NT-M | 3.9 × 10⁻⁹ |
| SEQ ID No.: 13 | CTFQKLAHQIYQ | 19-31 | MR-M | 4.5 × 10⁻¹⁰ |
| SEQ ID No.: 14 | CAPRNKISPQGY-NH₂ | C-40-50 | CT-M | 9 × 10⁻⁹ |

The following is a list of further obtained monoclonal antibodies:

TABLE 2
Further obtained monoclonal anti-ADM antibodies

| | | | | max inhibition |
|---|---|---|---|---|
| | | | | bioassay (%) |
| Target | Source | Clone number | Affinity (M) | (see example 2) |
| NT-M | Mouse | ADM/63 | 5.8 × 10⁻⁹ | 45 |
| | Mouse | ADM/364 | 2.2 × 10⁻⁸ | 48 |
| | Mouse | ADM/365 | 3.0 × 10⁻⁸ | |
| | Mouse | ADM/366 | 1.7 × 10⁻⁸ | |
| | Mouse | ADM/367 | 1.3 × 10⁻⁸ | |
| | Mouse | ADM/368 | 1.9 × 10⁻⁸ | |
| | Mouse | ADM/369 | 2.0 × 10⁻⁸ | |
| | Mouse | ADM/370 | 1.6 × 10⁻⁸ | |
| | Mouse | ADM/371 | 2.0 × 10⁻⁸ | |
| | Mouse | ADM/372 | 2.5 × 10⁻⁸ | |
| | Mouse | ADM/373 | 1.8 × 10⁻⁸ | |
| | Mouse | ADM/377 | 1.5 × 10⁻⁸ | |
| | Mouse | ADM/378 | 2.2 × 10⁻⁸ | |
| | Mouse | ADM/379 | 1.6 × 10⁻⁸ | |
| | Mouse | ADM/380 | 1.8 × 10⁻⁸ | |
| | Mouse | ADM/381 | 2.4 × 10⁻⁸ | |
| | Mouse | ADM/382 | 1.6 × 10⁻⁸ | |
| | Mouse | ADM/383 | 1.8 × 10⁻⁸ | |
| | Mouse | ADM/384 | 1.7 × 10⁻⁸ | |
| | Mouse | ADM/385 | 1.7 × 10⁻⁸ | |
| | Mouse | ADM/403 | 1.2 × 10⁻⁸ | |
| | Mouse | ADM/395 | 1.2 × 10⁻⁸ | |
| | Mouse | ADM/396 | 3.0 × 10⁻⁸ | |
| | Mouse | ADM/397 | 1.5 × 10⁻⁸ | |
| MR-M | Mouse | ADM/38 | 4.5 × 10⁻¹⁰ | 68 |
| MR-M | Mouse | ADM/39 | 5.9 × 10⁻⁹ | 72 |
| CT-M | Mouse | ADM/65 | 9.0 × 10⁻⁹ | 100 |
| CT-M | Mouse | ADM/66 | 1.6 × 10⁻⁸ | 100 |
| NT-H | Mouse | ADM/33 | 5.9 × 10⁻⁸ | 38 |
| NT-H | Mouse | ADM/34 | 1.6 × 10⁻⁸ | 22 |
| MR-H | Mouse | ADM/41 | 1.2 × 10⁻⁸ | 67 |
| MR-H | Mouse | ADM/42 | <1 × 10⁻⁸ | |
| MR-H | Mouse | ADM/43 | 2.0 × 10⁻⁹ | 73 |
| MR-H | Mouse | ADM/44 | <1 × 10⁻⁸ | |
| CT-H | Mouse | ADM/15 | <1 × 10⁻⁸ | |
| CT-H | Mouse | ADM/16 | 1.1 × 10⁻⁹ | 100 |
| CT-H | Mouse | ADM/17 | 3.7 × 10⁻⁹ | 100 |
| CT-H | Mouse | ADM/18 | <1 × 10⁻⁸ | |
| hADM | Phage display | ADM/A7 | <1 × 10⁻⁸ | |
| | Phage display | ADM/B7 | <1 × 10⁻⁸ | |
| | Phage display | ADM/C7 | <1 × 10⁻⁸ | |
| | Phage display | ADM/G3 | <1 × 10⁻⁸ | |
| | Phage display | ADM/B6 | <1 × 10⁻⁸ | |
| | Phage display | ADM/B11 | <1 × 10⁻⁸ | |
| | Phage display | ADM/D8 | <1 × 10⁻⁸ | |
| | Phage display | ADM/D11 | <1 × 10⁻⁸ | |
| | Phage display | ADM/G12 | <1 × 10⁻⁸ | |

Generation of antibody fragments by enzymatic digestion: The generation of Fab and F(ab)₂ fragments was done by enzymatic digestion of the murine full-length antibody NT-M. Antibody NT-M was digested using a) the pepsin-based F(ab)₂ Preparation Kit (Pierce 44988) and b) the papain-based Fab Preparation Kit (Pierce 44985). The fragmentation procedures were performed according to the instructions provided by the supplier. Digestion was carried out in case of F(ab)₂-fragmentation for 8 h at 37° C. The Fab-fragmentation digestion was carried out for 16 h, respectively.

Procedure for Fab Generation and Purification: The immobilized papain was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample were added to the spin column tube containing the equilibrated Immobilized Papain. Incubation time of the digestion reaction was done for 16 h on a tabletop rocker at 37° C. The column was centrifuged at 5000×g for 1 minute to separate digest from the Immobilized Papain. Afterwards the resin was washed with 0.5 ml PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 ml of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Coulter and Harris 1983. J. Immunol. Meth. 59, 199-203.; Lindner et al. 2010. Cancer Res. 70, 277-87; Kaufmann et al. 2010. PNAS. 107, 18950-5.; Chen et al. 2010. PNAS. 107, 14727-32; Uysal et al. 2009 J. Exp. Med. 206, 449-62; Thomas et al. 2009. J. Exp. Med. 206, 1913-27; Kong et al. 2009 J Cell Biol. 185, 1275-840).

Procedure for generation and purification of F(ab′)₂ Fragments: The immobilized Pepsin was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample were added to the spin column tube containing the equilibrated Immobilized Pepsin. Incubation time of the digestion reaction was done for 16 h on a tabletop rocker at 37° C. The column was centrifuged at 5000×g for 1 minute to separate digest from the Immobilized Papain. Afterwards the resin was washed with 0.5 mL PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 mL of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Mariani et al. 1991. Mol. Immunol. 28: 69-77; Beale 1987. Exp Comp Immunol 11:287-96; Ellerson et al. 1972. FEBS Letters 24(3):318-22; Kerbel and Elliot 1983. Meth Enzymol 93:113-147; Kulkarni et al. 1985. Cancer Immunol Immunotherapy 19:211-4; Lamovi 1986. Meth Enzymol 121:652-663; Parham et al. 1982. J Immunol Meth 53:133-73; Ravchaudhuri et al. 1985. Mol Immunol 22(9):1009-19; Rousseaux et al. 1980. Mol Immunol 17:469-82; Rousseaux et al. 1983. J Immunol Meth 64:141-6; Wilson et al. 1991. J Immunol Meth 138:111-9).

NT-H-Antibody Fragment Humanization: The antibody fragment was humanized by the CDR-grafting method (Jones et al. 1986. Nature 321, 522-525).

The following steps were done to achieve the humanized sequence:

Total RNA extraction: Total RNA was extracted from NT-H hybridomas using the Qiagen kit. First-round RT-PCR: QIAGEN® OneStep RT-PCR Kit (Cat No. 210210) was used. RT-PCR was performed with primer sets specific for the heavy and light chains. For each RNA sample, 12 individual heavy chain and 11 μlight chain RT-PCR reactions were set up using degenerate forward primer mixtures covering the leader sequences of variable regions. Reverse primers are located in the constant regions of heavy and light chains. No restriction sites were engineered into the primers.

Reaction Setup: 5× QIAGEN® OneStep RT-PCR Buffer 5.0 μl, dNTP Mix (containing 10 mM of each dNTP) 0.8 μl, Primer set 0.5 μl, QIAGEN® OneStep RT-PCR Enzyme Mix 0.8 μl, Template RNA 2.0 μl, RNase-free water to 20.0 μl, Total volume 20.0 μl PCR condition: Reverse transcription: 50° C., 30 min; Initial PCR activation: 95° C., 15 min Cycling: 20 cycles of 94° C., 25 sec; 54° C., 30 sec; 72° C., 30 sec; Final extension: 72° C., 10 min Second-round semi-nested PCR: The RT-PCR products from the first-round reactions were further amplified in the second-round PCR. 12 individual heavy chain and 11 μlight chain RT-PCR reactions were set up using semi-nested primer sets specific for antibody variable regions.

Reaction Setup: 2× PCR mix 10 μl; Primer set 2 μl; First-round PCR product 8 μl; Total volume 20 μl; Hybridoma Antibody Cloning Report PCR condition: Initial denaturing of 5 min at 95° C.; 25 cycles of 95° C. for 25 sec, 57° C. for 30 sec, 68° C. for 30 sec; Final extension is 10 min 68° C.

After PCR was finished, PCR reaction samples were run onto agarose gel to visualize DNA fragments amplified. After sequencing more than 15 cloned DNA fragments amplified by nested RT-PCR, several mouse antibody heavy and light chains have been cloned and appear correct. Protein sequence alignment and CDR analysis identifies one heavy chain and one light chain. After alignment with homologous human framework sequences the resulting humanized sequence for the variable heavy chain is the following: see FIG. 5. As the amino acids on positions 26, 40 and 55 in the variable heavy chain and amino acid on position 40 in the variable light are critical to the binding properties, they may be reverted to the murine original. The resulting candidates are depicted below. (Padlan 1991. Mol. Immunol. 28, 489-498; Harris and Bajorath.1995. Protein Sci. 4, 306-310).

Annotation for the antibody fragment sequences (SEQ ID No.: 24-31; 32 and 33): bold and underline are the CDR 1, 2, 3 in chronologically arranged; italic are constant regions; hinge regions are highlighted with bold letters.

(AM-VH-C)
SEQ ID No.: 24
QVQLQQSGAELMKPGASVKISCKATGYTFSRYWIEWVKQRPGHGLEWIGEILPGSGSTNYN
EKFKGKATITADTSSNTAYMQLSSLTSEDSAVYYCTEGYEYDGFDYWGQGTTLTVSSASTKGPSVF
PLAPSSKSTSGGTAALGCLVKDYFPEPVTVSWNSGALTSGVHTFPAVLQSSGLYSLSSVVTVPSSSLGT
QTYICNVNHKPSNTKVDKRVEPK
(AM-VH1)
SEQ ID No.: 25
QVQLVQSGAEVKKPGSSVKVSCKASGYTFSRYWISWVRQAPGQGLEWMGRILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VH2-E40)
SEQ ID No.: 26
QVQLVQSGAEVKKPGSSVKVSCKASGYTFSRYWIEWVRQAPGQGLEWMGRILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VH3-T26-E55)
SEQ ID No.: 27
QVQLVQSGAEVKKPGSSVKVSCKATGYTFSRYWISWVRQAPGQGLEWMGEILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VH4-T26-E40-E55)
SEQ ID No.: 28
QVQLVQSGAEVKKPGSSVKVSCKATGYTFSRYWIEWVRQAPGQGLEWMGEILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VL-C)
SEQ ID No.: 29
DVLLSQTPLSLPVSLGDQATISCRSSQSIVYSNGNTYLEWYLQKPGQSPKLLIYRVSNRFSGVP
DRFSGSGSGTDFTLKISRVEAEDLGVYYCFQGSHIPYTFGGGTKLEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGEC
(AM-VL1)
SEQ ID No.: 30
DVVMTQSPLSLPVTLGQPASISCRSSQSIVYSNGNTYLNWFQQRPGQSPRRLIYRVSNRDSGV
PDRFSGSGSGTDFTLKISRVEAEDVGVYYCFQGSHIPYTFGQGTKLEIKRTVAAPSVFIFPPSD
EQLKSGTASVVCLLNNFYPREAKVQWKVDNALQSGNSQESVTEQDSKDSTYSLSSTLTLSKA
DYEKHKVYACEVTHQGLSSPVTKSFNRGEC
(AM-VL2-E40)
SEQ ID No.: 31
DVVMTQSPLSLPVTLGQPASISCRSSQSIVYSNGNTYLEWFQQRPGQSPRRLIYRVSNRDSGV
PDRFSGSGSGTDFTLKISRVEAEDVGVYYCFQGSHIPYTFGQGTKLEIKRTVAAPSVFIFPPSD
EQLKSGTASVVCLLNNFYPREAKVQWKVDNALQSGNSQESVTEQDSKDSTYSLSSTLTLSKA
DYEKHKVYACEVTHQGLSSPVTKSFNRGEC
(Enibarcimab heavy chain)
SEQ ID No: 32
QVQLVQSGAEVKKPGSSVKVSCKASGYTFSRYWIEWVRQAPGQGLEWIGEILPGSGSTNYN
QKFQGRVTITADTSTSTAYMELSSLRSEDTAVYYCTEGYEYDGFDYWGQGTTVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALH
NHYTQKSLSLSPGK
SEQ ID No.: 33 (Enibarcimab light chain)
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGEC

### Example 2—Effect of Selected Anti-ADM-Antibodies on ADM-Bioactivity

The effect of selected ADM-antibodies on ADM-bioactivity was tested in a human recombinant Adrenomedullin receptor cAMP functional assay (Adrenomedullin Bioassay).

Testing of antibodies targeting human or mouse adrenomedullin in human recombinant Adrenomedullin receptor cAMP functional assay (Adrenomedullin Bioassay)

Materials: Cell line CHO-K1, Receptor Adrenomedullin (CRLR+RAMP3), Receptor Accession Number Cell line: CRLR: U17473; RAMP3: AJ001016

CHO-K1 cells expressing human recombinant adrenomedullin receptor (FAST-027C) grown prior to the test in media without antibiotic were detached by gentle flushing with PBS-EDTA (5 mM EDTA), recovered by centrifugation and resuspended in assay buffer (KRH: 5 mM KCl, 1.25 mM MgSO₄, 124 mM NaCl, 25 mM HEPES, 13.3 mM Glucose, 1.25 mM KH₂PO₄, 1.45 mM CaCl₂), 0.5 g/l BSA).

Dose response curves were performed in parallel with the reference agonists (hADM or mADM).

Antagonist test (96well): For antagonist testing, 6 μl of the reference agonist (human (5.63 nM) or mouse (0.67 nM) adrenomedullin) was mixed with 6 μl of the test samples at different antagonist dilutions; or with 6 μl buffer. After incubation for 60 min at room temperature, 12 μl of cells (2,500 cells/well) were added. The plates were incubated for 30 min at room temperature. After addition of the lysis buffer, percentage of DeltaF will be estimated, according to the manufacturer specification, with the HTRF kit from Cis-Bio International (cat n° 62AM2 PEB) hADM 22-52 was used as reference antagonist.

Antibodies Testing cAMP-HTRF Assay

The anti-h-ADM antibodies (NT-H, MR-H, CT-H) were tested for antagonist activity in human recombinant adrenomedullin receptor (FAST-027C) cAMP functional assay in the presence of 5.63 nM Human ADM 1-52, at the following final antibody concentrations: 100 μg/ml, 20 μg/ml, 4 μg/ml, 0.8 μg/ml, 0.16 μg/ml.

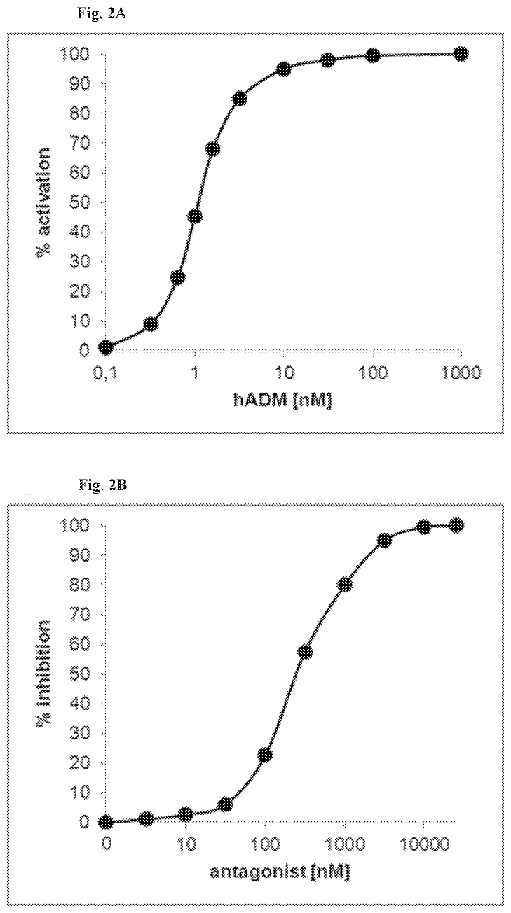

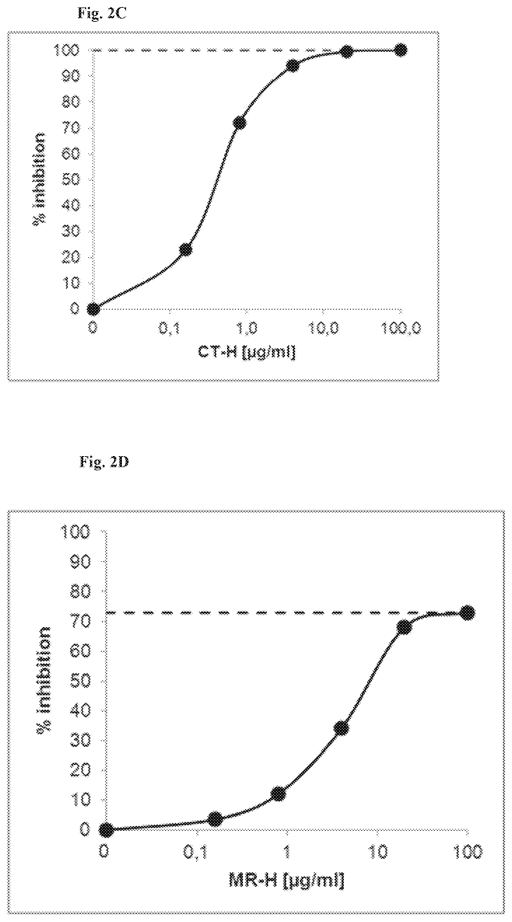

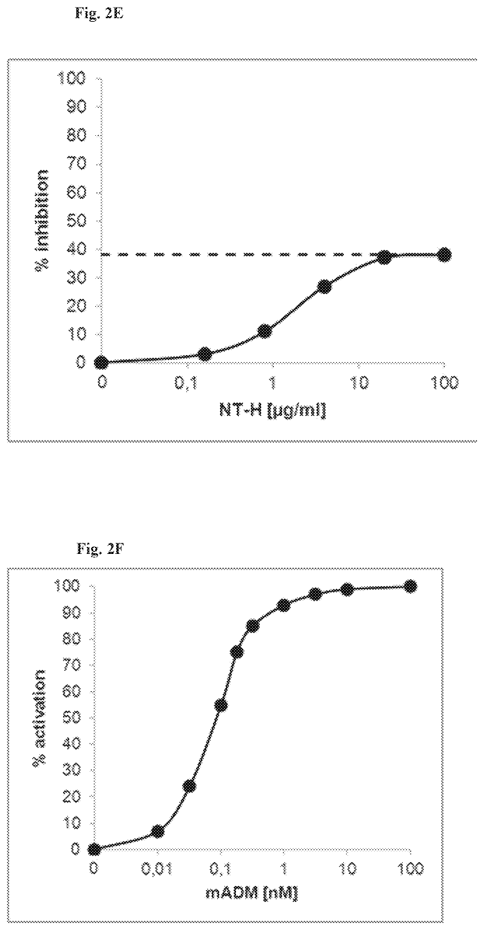

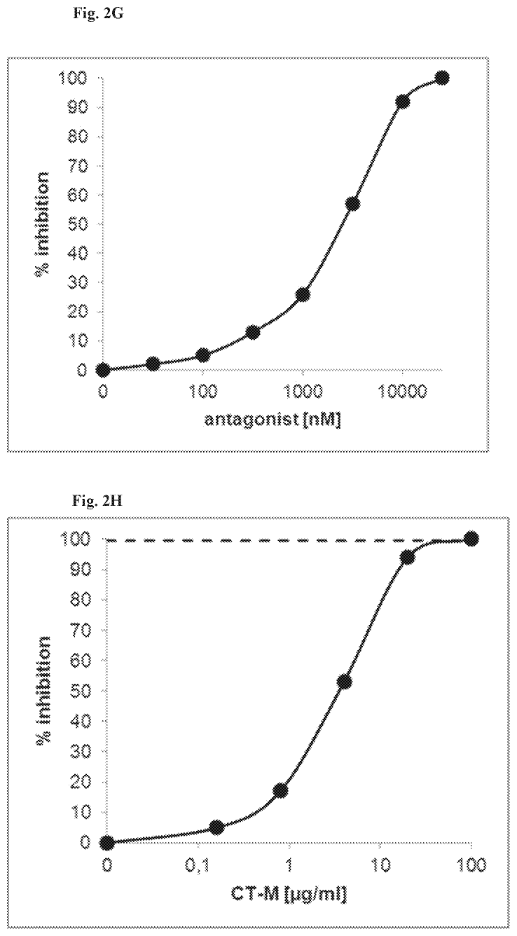

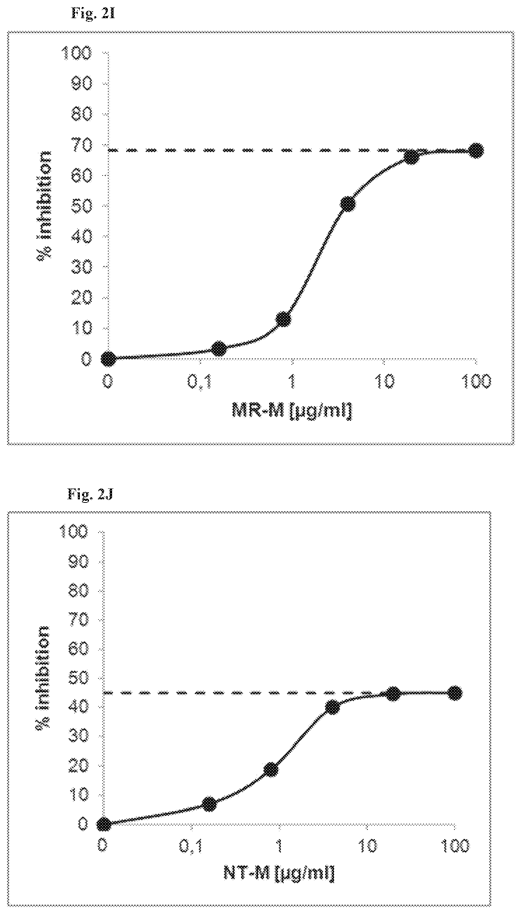

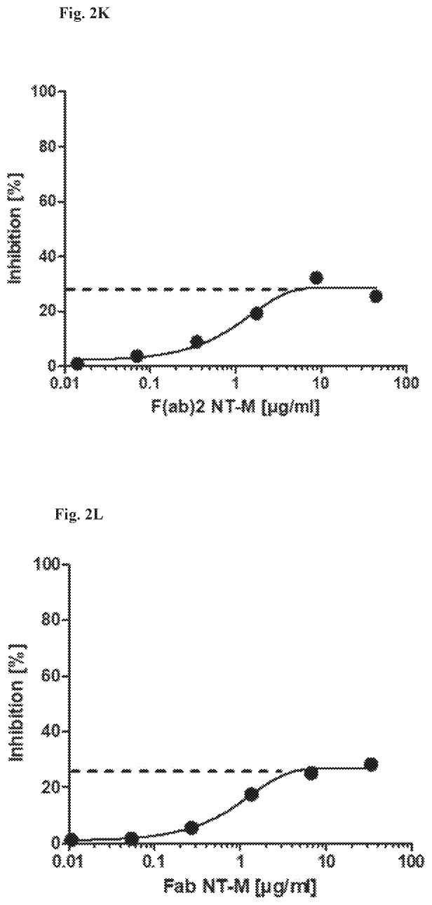

The anti-m-ADM antibodies (NT-M, MR-M, CT-M) were tested for antagonist activity in human recombinant ADM receptor (FAST-027C) cAMP functional assay in the presence of 0.67 nM Mouse ADM 1-50, at the following final antibody concentrations: 100 μg/ml, 20 μg/ml, 4 μg/ml, 0.8 μg/ml, 0.16 μg/ml. Data were plotted relative inhibition vs. antagonist concentration (see FIGS. 2A-2L). The maximal inhibition by the individual antibody is given in table 3.

TABLE 3
Maximal inhibition of bio-ADM activity

| | Maximal inhibition of ADM bioactivity |
|---|---|
| Antibody | (ADM-Bioassay) (%) |
| NT-H | 38 |
| MR-H | 73 |
| CT-H | 100 |
| NT-M FAB | 26 |
| NT-M FAB2 | 28 |
| NT-M | 45 |
| MR-M | 66 |
| CT-M | 100 |
| Non specific mouse IgG | 0 |

### Example 3—Stabilization of hADM by the Anti-ADM Antibody

The stabilizing effect of human ADM by human ADM antibodies was tested using a hADM immunoassay.

### Immunoassay for the Quantification of Human Adrenomedullin

The technology used was a sandwich coated tube luminescence immunoassay, based on Acridinium ester labelling.

Labelled compound (tracer): 100 μg (100 μl) CT-H (1 mg/ml in PBS, pH 7.4, AdrenoMed AG Germany) was mixed with 10 μl Acridinium NHS-ester (1 mg/ml in acetonitrile, InVent GmbH, Germany) (EP 0353971) and incubated for 20 min at room temperature. Labelled CT-H was purified by Gel-filtration HPLC on Bio-Sil® SEC 400-5 (Bio-Rad Laboratories, Inc., USA) The purified CT-H was diluted in (300 mmol/L potassium phosphate, 100 mmol/L NaCl, 10 mmol/L Na-EDTA, 5 g/L Bovine Serum Albumin, pH 7.0). The final concentration was approx. 800.000 relative light units (RLU) of labelled compound (approx. 20 ng labeled antibody) per 200 pL. Acridiniumester chemiluminescence was measured by using an AutoLumat LB 953 (Berthold Technologies GmbH & Co. KG).

Solid phase: Polystyrene tubes (Greiner Bio-One International AG, Austria) were coated (18 h at room temperature) with MR-H (AdrenoMed AG, Germany) (1.5 μg MR-H/0.3 mL 100 mmol/L NaCl, 50 mmol/L TRIS/HCl, pH 7.8). After blocking with 5% bovine serum albumin, the tubes were washed with PBS, pH 7.4 and vacuum dried.

Calibration: The assay was calibrated, using dilutions of hADM (BACHEM AG, Switzerland) in 250 mmol/L NaCl, 2 g/L Triton X-100, 50 g/L Bovine Serum Albumin, 20 tabs/L Protease Inhibitor Cocktail (Roche Diagnostics AG, Switzerland).

hADM Immunoassay: 50 μl of sample (or calibrator) was pipetted into coated tubes, after adding labeled CT-H (200 μl), the tubes were incubated for 4 h at 4° C. Unbound tracer was removed by washing 5 times (each 1 ml) with washing solution (20 mM PBS, pH 7.4, 0.1% Triton X-100).

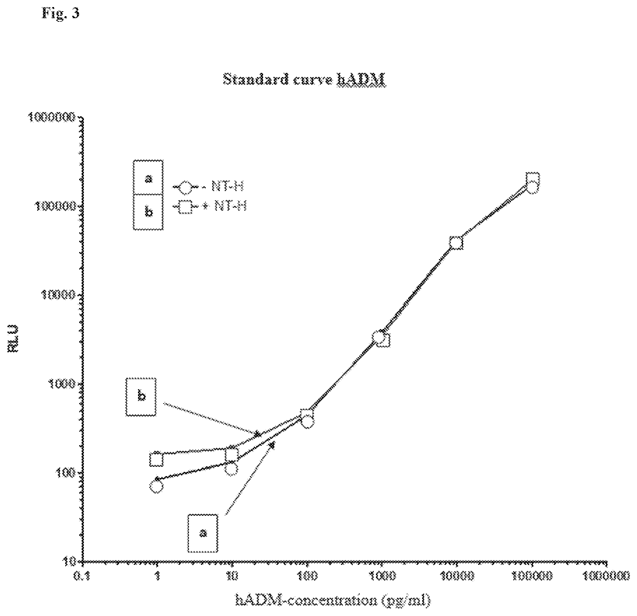

Tube-bound chemiluminescence was measured by using the LB 953: FIG. 3 shows a typical hADM dose/signal curve. And an hADM dose signal curve in the presence of 100 μg/mL antibody NT-H. NT-H did not affect the described hADM immunoassay.

Stability of human Adrenomedullin: Human ADM was diluted in human Citrate plasma (final concentration 10 nM) and incubated at 24° C. At selected time points, the degradation of hADM was stopped by freezing at −20° C. The incubation was performed in absence and presence of NT-H (100 μg/ml). The remaining hADM was quantified by using the hADM immunoassay described above.

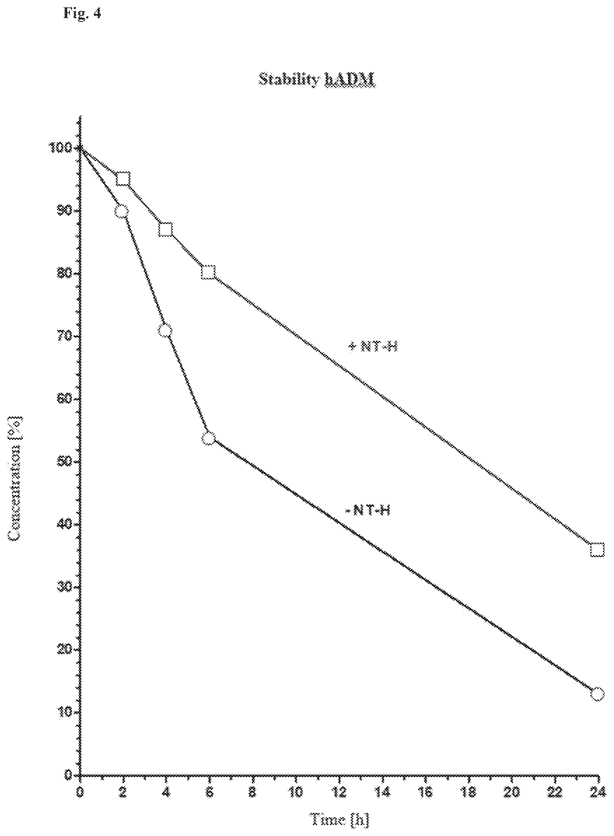

FIG. 4 shows the stability of hADM in human plasma (citrate) in absence and in the presence of NT-H antibody. The half-life of hADM alone was 7.8 h and in the presence of NT-H, the half-life was 18.3 h. (2.3 times higher stability).

### Example 4—Administration of NT-H Antibodies in Healthy Humans

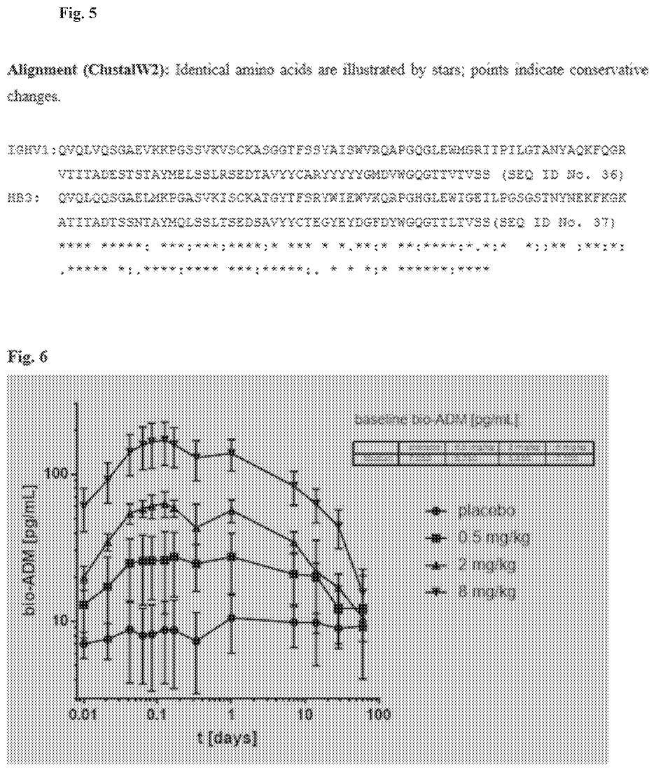

The study was conducted in healthy male subjects as a randomized, double-blind, placebo-controlled, study with single escalating doses of NT-H antibody administered as intravenous (i.v.) infusion in 3 sequential groups of 8 healthy male subjects each (1st group 0.5 mg/kg, 2nd group 2 mg/kg, 3rd group 8 mg/kg) of healthy male subjects (n=6 active, n=2 μlacebo for each group). The main inclusion criteria were written informed consent, age 18-35 years, agreement to use a reliable way of contraception and a BMI between 18 and 30 kg/m². Subjects received a single i.v. dose of NT-H antibody (0.5 mg/kg; 2 mg/kg; 8 mg/kg) or placebo by slow infusion over a 1-hour period in a research unit. The baseline ADM-values in the 4 groups did not differ. Median ADM values were 7.1 μg/mL in the placebo group, 6.8 pg/mL in the first treatment group (0.5 mg/kg), 5.5 μg/mL in second treatment group (2 mg/kg) and 7.1 pg/mL in the third treatment group (8 mg/mL). The results show that ADM-values rapidly increased within the first 1.5 hours after administration of NT-H antibody in healthy human individuals, then reached a plateau and slowly declined (FIG. 6).

### Example 5—Measurement of Bio-ADM and ADM-Gly

Quantification of bio-ADM was conducted using the sphingotest bio-ADM assay as described elsewhere (Weber, J. et al. Sandwich Immunoassay for Bioactive Plasma Adrenomedullin. J. Appl. Lab. Med. An AACC Publ. 2, 222-233 (2017)): 96-well high binding polystyrene microtiter plates (Greiner Bio-One International AG) were coated (18 h at 20° C.) with monoclonal anti-ADM antibody, directed towards amino-acids 21-32 of bio-ADM (SEQ ID No.: 10) (HAM2203, 1 g/0.2 mL per well in 50 mM Tris-HCl, 100 mM NaCl, pH 7.8). After blocking with 30 g/L Karion, 5 g/L BSA (protease free), 6.5 mmol/L monopotassium phosphate, 3.5 mmol/L sodium dihydrogen phosphate (pH 6.5), the plates were vacuum-dried.

100 μL of samples/calibrators were pipetted into coated microtiter plates. Afterwards 150 μL of MACN labelled tracer antibody (HAM2302, directed towards the amidated C-terminus of bio-ADM (SEQ ID No.: 11) the microtiter plates were incubated for 1 h at 22° C. under agitation at 600 rpm. Unbound tracer was removed by washing 5 times (each 350 μL per well) with washing solution (20 mM PBS, 1 g/L Triton X-100, pH 7.4). Well-bound chemiluminescence was measured for 1 s per well by using the Centro LB 960 microtiter plate luminescence reader (Berthold Technologies). The assay was calibrated using dilutions of synthetic human bio-ADM (American Peptide Company). The lowest calibrator did not contain bio-ADM, but a concentration of 2 μg/mL was assigned to facilitate logarithmic evaluation. The calibrators were lyophilized in 20 mmol K₂PO4, 6 mmol/L Na-EDTA, 5 g/L BSA, 100 μmol/L leupeptin, 50 μmol/L amastatin, 10 g/mL of anti-N-terminal antibody HAM1101, pH 8.0, and reconstituted in H₂O before use.

The molar concentration of the bio-ADM calibrator peptide preparation may be calculated from the mass concentration of the peptide preparation using the molecular weight of bio-ADM, wherein the mass concentration results from dissolving a specific mass of the bio-ADM preparation in a defined volume of a solvent. The molecular weight of the bio-ADM peptide is defined as the sum of weights (in Dalton) of all Atoms in the peptide. Thereby hydrogen has the weight of 1 Da. The molecular weight of a peptide preparation may vary from the calculated molecular weight in dependence on the used counter ion in the synthesis of the peptide and the number of basic amino acids and free amides within of the peptide sequence the counter ion binds to. A counter ion might be, but is not limited to, trifluoroacetic acid (TFA) with the molecular weight of 114.02 Da. The molecular weight (MW) of a synthetic peptide may be calculated as follows: MW_{peptide}=(sum of all atoms in the peptide sequence)+x*MW_{counterion}, wherein x is the number of free amides including basic amino acids within of peptides sequence. The molecular weight of bio-ADM may be additionally determined using mass spectrometry, in particular using matrix-assisted laser desorption/ionization time-of-flight mass spectrometry (MALDI-TOF MS). A detailed procedure for the analysis of peptides containing disulfide bonds, as is the case in bio-ADM, using MALDI-TOF MS is described in (Huwiler K G, Mosher D F, Vestling M M. Optimizing the MALDI-TOF-MS observation ofpeptides containing disulfide bonds. JBiomol Tech. 2003 December; 14(4):289-97).

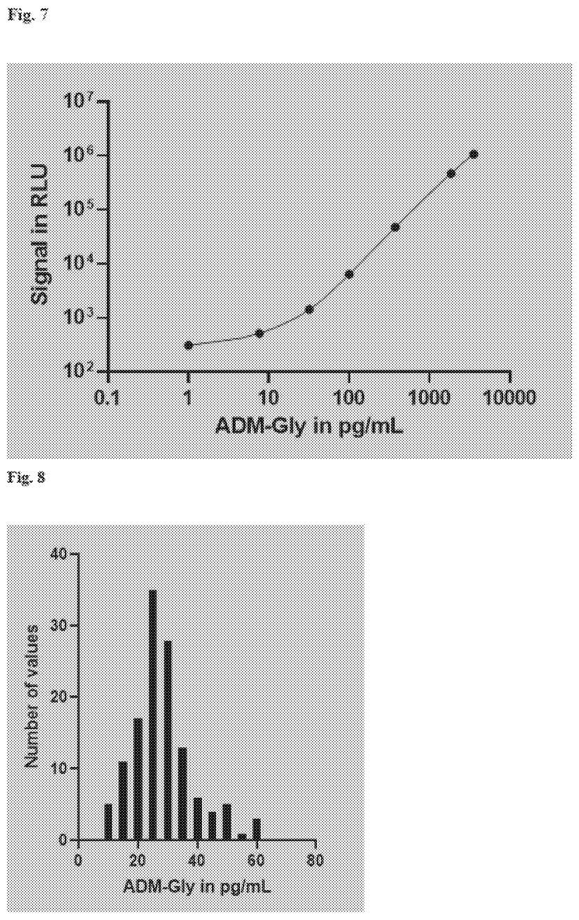

Quantification of ADM-Gly was conducted as follows: 96-well high binding polystyrene microtiter plates (Greiner Bio-One International AG) were coated (18 h at 20° C.) with monoclonal anti-ADM antibody, directed towards amino-acids 21-32 of ADM-Gly (SEQ ID No.: 10) (HAM2203, 1 g/0.2 mL per well in 50 mM Tris-HCl, 100 mM NaCl, pH 7.8). After blocking with 30 g/L Karion, 5 g/L BSA (protease free), 6.5 mmol/L monopotassium phosphate, 3.5 mmol/L sodium dihydrogen phosphate (pH 6.5), the plates were vacuum-dried. 50 μL of samples/calibrators were pipetted into coated microtiter plates. Afterwards 200 μL of MACN labelled tracer antibody (AK835/G4, directed towards the glycine extended C-terminus of ADM-Gly (SEQ ID No.: 34). AK835/G4 had no cross-reactivity with bio-ADM) the microtiter plates were incubated for 18 h at 4° C. under agitation at 600 rpm. Unbound tracer was removed by washing 5 times (each 350 μL per well) with washing solution (20 mM PBS, 1 g/L Triton X-100, pH 7.4). Well-bound chemiluminescence was measured for 1 s per well by using the Centro LB 960 microtiter plate luminescence reader (Berthold Technologies). The assay was calibrated using dilutions of synthetic human ADM-Gly (SEQ ID No.: 6) (Peptides and Elephants, Hennigsdorf, Germany). The lowest calibrator did not contain ADM-Gly, but a concentration of 1 μg/mL was assigned to facilitate logarithmic evaluation. The calibrators were lyophilized in 20 mmol K2PO4,6 mmol/L Na-EDTA, 5 g/L BSA, 100 μmol/L leupeptin, 50 μmol/L amastatin, 10 g/mL of anti-N-terminal antibody HAMI 101, pH 8.0, and reconstituted in H₂O before use. A typical standard curve is shown in FIG. 7. A normal distribution of ADM-Gly from n=128 self-reported healthy individuals is shown in FIG. 8. The median concentration of ADM-Gly in this cohort was 27.1 μg/mL, the 25^{th} percentile was 22 μg/mL, the 75^{th} percentile was 32.7. pg/mL, the 5^{th} percentile was 13.1 μg/mL, and the 95^{th} percentile was 51.3 pg/mL.

### Example 6

A cohort of seventeen patients with advanced relapsed and/or refractory lymphoma or leukemia was treated with CAR T-cells following a established procedure as described (Ayala Ceja et al., 2024). Blood samples were collected immediately prior to the administration of T-cells as well as after the treatment, triggered by clinical events including diagnosis and treatment of complications related to a cytokine release syndrome (CRS). CRS was defined and graded according to American Society for Transplantation and Cellular Therapy (ASTCT) guidelines (D. W. Lee et al., 2019). The grading system has defined a range of 4 grades, with grade 4 being the most severe conditions. Blood samples for measurement of bio-ADM and ADM-Gly were gained Just before the administration of T-cells in the frame of CAR T-cell therapy (baseline), and then at several time points in the days following the CAR-T cell therapy. Baseline concentrations of bio-ADM and ADM-Gly are shown in Table 4.

TABLE 4
Baseline bio-ADM- and ADM-Gly levels of patients
treated with CAR T-cells, and their CRS grading
resulting from the CAR T-cell therapy.

| Patient # | bio-ADM [pg/mL] | ADM-Gly [pg/mL] | CRS Grading |
|---|---|---|---|
| 1 | 54.3 | 40.7 | 2 |
| 2 | 10.4 | 27.4 | 3 |
| 3 | 114.5 | 336.5 | 2 |
| 4 | 17.0 | 66.5 | 1 |
| 5 | 26.2 | 41.7 | 1 |
| 6 | 08.3 | 18.9 | 0 |
| 7 | 12.5 | 21.7 | 2 |
| 8 | 35.8 | 101.6 | 2 |
| 9 | 16.3 | 37.4 | 0 |
| 10 | 15.6 | 30.5 | 0 |
| 11 | 96.1 | 85.3 | 3 |
| 12 | 51.5 | 142.4 | 3 |
| 13 | 155.8 | 192.0 | 4 |
| 14 | 17.5 | 46.7 | 0 |
| 15 | 14.8 | 48.0 | 0 |
| 16 | 32.4 | 37.1 | 3 |
| 17 | 14.4 | 41.2 | 3 |

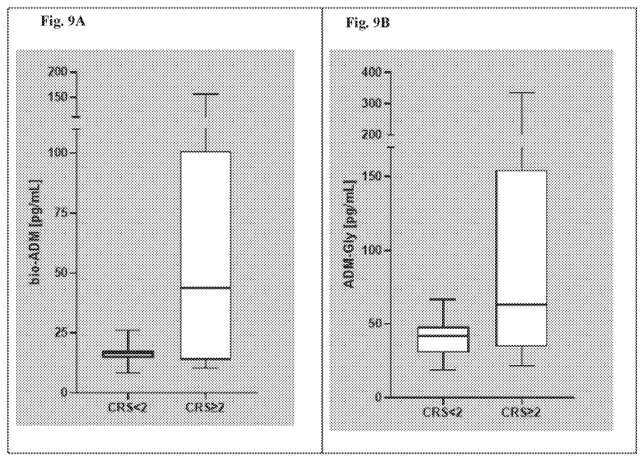

When the baseline concentrations of bio-ADM and ADM-Gly were separated in groups of patients who after the start of the CAR T-cell therapy either developed severe side-effects (defined as CRS score ≥2) or did not or only very moderately develop such effects (defined as CRS <2), it surprisingly turned out that patients developing severe side-effects presented with strongly elevated concentrations of bio-ADM and ADM-Gly already prior to start of the administration of T-cells in the frame of CAR T-cell therapy (median: 43.7 μg/ml and 63.3 μg/mL, respectively) compared to those patients who did not or only very moderately develop such effects (median: 16.3 μg/mL and 41.7 μg/mL, respectively) (FIGS. 9A and 9B). This observation demonstrates that measurement of bio-ADM, ADM-Gly or related peptides is suitable to detect prior to administration of CAR T-cells in the frame of a CAR T-cell therapy the risk of development of severe side effects. Elevation of bio-ADM or ADM-Gly is considered an indication of endothelial dysfunction (van Lier et al., 2020). Thus, administration of an anti-ADM antibody suitable for the treatment of endothelial dysfunction in cancer patients with elevated levels of bio-ADM or ADM-Gly prior to start of administration of CAR T-cells in the frame of a CAR T-cell therapy would be an ideal tool to prevent the development of severe side effects eventually developing after administration of CAR T-cells.

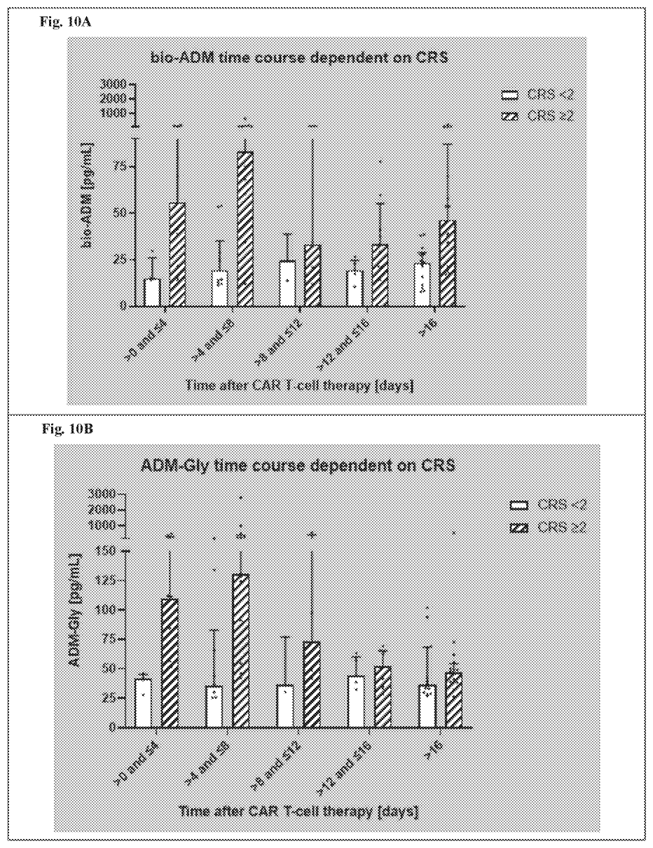

Both markers, bio-ADM and ADM-Gly, were also measured in the follow-up of the patients. As blood draws were not taken at the exact same time points after administration of T-cells in the frame of CAR-T cell therapy for all patients, data from bio-ADM- and ADM-Gly measurements were grouped in time intervals of four days, e.g. >0 and ≤4 days, >4 and ≤8 days, >8 and ≤12 days, >12 and ≤16 days, >16 days, after administration of CAR T-cells in the frame of CAR T-cell therapy. For both markers, bio-ADM and ADM-Gly, it was observed that concentrations increased after administration of T-cells in the frame of CAR T-cell therapy until the time interval >4 and ≤8 days, followed by a decline, only for patients who developed severe side-effects (defined as CRS score ≥2) (FIGS. 10A and 10B). In contrast, patients who did not or only very moderately develop such effects (defined as CRS <2), exhibited constant low levels of bio-ADM and ADM-Gly over the entire observation period. Thus, measurement of bio-ADM, ADM-Gly or related peptides is suitable to monitor endothelial dysfunction associated with the severity of side-effects arising from CAR T-cell therapy.

SEQUENCES

SEQ ID No.: 1-PreproADM, amino acids 1 to 185
MKLVSVALMYLGSLAFLGADTARLDVASEFRKKWNKWALSRGKRELRMSSSYPTGLADVK
AGPAQTLIRPQDMKGASRSPEDSSPDAARIRVKRYRQSMNNFQGLRSFGCRFGTCTVQKLAH
QIYQFTDKDKDNVAPRSKISPQGYGRRRRRSLPEAGPGRTLVSSKPQAHGAPAPPSGSAPHFL
SEQ ID No.: 2-Proadrenomedullin, amino acids 22-185 of preproADM
ARLDVASEF RKKWNKWALS RGKRELRMSS SYPTGLADVKAGPAQTLIRP QDMKGASRSP
EDSSPDAARIRVKRYRQSMN NFQGLRSFGCRFGTCTVQKLAHQIYQFTDK DKDNVAPRSK
ISPQGYGRRRRRSLPEAGPGRTLVSSKPQAHGAPAPPSGSAPHFL
SEQ ID No.: 3-PAMP-Gly, amino acids 22-42 of pre-proADM SEQ ID No. 1
ARLDVASEFRKKWNKWALSRG
SEQ ID No. 4-PAMP-NH₂, amino acids 22-41 of pre-proADM SEQ ID No. 1
ARLDVASEFRKKWNKWALSR
SEQ ID No.: 5-MR-proADM, amino acids 45-92 of pre-proADM SEQ ID No. 1
ELRMSSSYPTGLADVKAGPAQTLIRPQDMKGASRSPEDSSPDAARIRV
SEQ ID No.: 6-ADM-Gly, amino acids 95-147 of pre-proADM SEQ ID No. 1
YRQSMNNFQG LRSFGCRFGTCTVQKLAHQIYQFTDKDKDNVAPRSKISPQGYG
SEQ ID No.: 7-mature ADM, amino acids 95-146 of pre-proADM SEQ ID No. 1
YRQSMNNFQGLRSFGCRFGTCTVQKLAHQIYQFTDKDKDNVAPRSKISPQGY
SEQ ID No.: 8-CT-proADM, amino acids 148-185 of pre-proADM SEQ ID No. 1
RRRRRSLPEAGPGRTLVSSKPQAHGAPAPPSGSAPHFL
SEQ ID No. 9-N-terminal part, amino acids 1-21 of ADM
YRQSMNNFQGLRSFGCRFGTC
SEQ ID No.: 10-MR-H, amino acids 21-32 of human ADM
CTVQKLAHQIYQ
SEQ ID No.: 11-CT-H-NH₂, amino acids 42-52 of human ADM
CAPRSKISPQGY
SEQ ID No.: 12-NT-M, amino acids 1-19 of murine ADM
YRQSMNQGSRSNGCRFGTC
SEQ ID No.: 13-MR-M, amino acids 19-31 of murine ADM
CTFQKLAHQIYQ
SEQ ID No.: 14-CT-M-NH₂, amino acids 40-50 of murine ADM
CAPRNKISPQGY
SEQ ID No.: 15-C-terminal portion of ADM, amino acids 43-52 of ADM
PRSKISPQGY-NH₂
SEQ ID No.: 16-N-terminal part, amino acids 1-14 of ADM
YRQSMNNFQGLRSF
SEQ ID No.: 17-N-terminal part, amino acids 1-10 of ADM
YRQSMNNFQG
SEQ ID No.: 18-N-terminal part, amino acids 1-6 of ADM
YRQSMN
SEQ ID No.: 19-Heavy chain, CDR1
GYTFSRYW
SEQ ID No.: 20-Heavy chain, CDR2
ILPGSGST
SEQ ID No.: 21-Heavy chain, CDR3
TEGYEYDGFDY
SEQ ID No.: 22-Light chain, CDR1
QSIVYSNGNTY
Light chain CDR2: (not assigned a SEQ ID No)
RVS
SEQ ID No.: 23-Light chain, CDR3
FQGSHIPYT.
SEQ ID No.: 24-AM-VH-C
QVQLQQSGAELMKPGASVKISCKATGYTFSRYWIEWVKQRPGHGLEWIGEILPGSGSTNYNE
KFKGKATITADTSSNTAYMQLSSLTSEDSAVYYCTEGYEYDGFDYWGQGTTLTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKRVEPK
SEQ ID No.: 25-AM-VH1
QVQLVQSGAEVKKPGSSVKVSCKASGYTFSRYWISWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 26-AM-VH2-E40
QVQLVQSGAEVKKPGSSVKVSCKASGYTFSRYWIEWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 27-AM-VH3-T26-E55
QVQLVQSGAEVKKPGSSVKVSCKATGYTFSRYWISWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 28-AM-VH4-T26-E40-E55
QVQLVQSGAEVKKPGSSVKVSCKATGYTFSRYWIEWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 29-AM-VL-C
DVLLSQTPLSLPVSLGDQATISCRSSQSIVYSNGNTYLEWYLQKPGQSPKLLIYRVSNRFSGVP
DRFSGSGSGTDFTLKISRVEAEDLGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID No.: 30 (AM-VL1)
DVVMTQSPLSLPVTLGQPASISCRSSQSIVYSNGNTYLNWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID No.: 31-AM-VL2-E40
DVVMTQSPLSLPVTLGQPASISCRSSQSIVYSNGNTYLEWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC.
SEQ ID No.: 32-Enibarcimab heavy chain
QVQLVQSGAEVKKPGSSVKVSCKASGYTFSRYWIEWVRQAPGQGLEWIGEILPGSGSTNYNQ
KFQGRVTITADTSTSTAYMELSSLRSEDTAVYYCTEGYEYDGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNH
YTQKSLSLSPGK
SEQ ID No.: 33-Enibarcimab light chain
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID No.: 34-C-terminal portion of ADM-Gly
CAPRSKISPQGYG
SEQ ID No. 35: Enibarcimab heavy chain Fab fragment
QVQLVQSGAEVKKPGSSVKVSCKASGYTFSRYWIEWVRQAPGQGLEWIGEILPGSGSTNYNQ
KFQGRVTITADTSTSTAYMELSSLRSEDTAVYYCTEGYEYDGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTH
SEQ ID No. 36-IGHV1-69*11 (see FIG. 5)
QVQLVQSGAEVKKPGSSVKVSCKASGGTFSSYAISWVRQAPGQGLEWMGRIIPILGTANYAQ
KFQGRVTITADESTSTAYMELSSLRSEDTAVYYCARYYYYYGMDVWGQGTTVTVSS
SEQ ID No. 37-HB3 (see FIG. 5)
QVQLQQSGAELMKPGASVKISCKATGYTFSRYWIEWVKQRPGHGLEWIGEILPGSGSTNYNE
KFKGKATITADTSSNTAYMQLSSLTSEDSAVYYCTEGYEYDGFDYWGQGTTLTVSS

1. Anti-ADM antibody or anti-ADM antibody fragment or anti-ADM non-Ig scaffold for use in therapy or prevention of side effects of immune effector cell administration to a cancer patient in the frame of an immune effector cell therapy (IECT), wherein said anti-ADM antibody or anti-ADM antibody fragment or anti-ADM non-Ig scaffold is administered prior to or after administration of said immune effector cells to said patient.

2. Anti-ADM antibody or anti-ADM antibody fragment or anti-ADM non-Ig scaffold for use according to claim 1, wherein said side effects of immune effector cell administration in the frame of an immune effector cell therapy (IECT) are associated with endothelial dysfunction.

3. Anti-ADM antibody or anti-ADM antibody fragment or anti-ADM non-Ig scaffold for use according to claim 1, wherein said anti-ADM antibody or anti-ADM antibody fragment or anti-ADM non-Ig scaffold is used for reducing immune effector cell therapy side effects.

4. Anti-ADM antibody or anti-ADM antibody fragment or anti-ADM non-Ig scaffold for use according to claim 1, wherein said immune effector cell therapy (IECT) is selected from the group comprising Chimeric Antigen Receptor T-cell (CAR-T) therapy, natural killer cell (NK) therapy, chimeric antigen receptor natural killer cell (CAR-NK) therapy, T cell receptor-engineered T cell (TCR T) therapy, tumor-infiltrating T cell (TIT)), and cytokine-induced killer cell (CIK) therapy.

5. Anti-ADM antibody or anti-ADM antibody fragment or anti-ADM non-Ig scaffold for use according to claim 1, wherein said therapy comprises the administration of further medication for the treatment of immune effector cell therapy side effects, preferably wherein said further medication is selected from the group comprising antihistamines, non-steroidal anti-inflammatory drugs (NSAIDs), immunosuppressive drugs, vasopressors, fluids and oxygen, preferably wherein said immunosuppressive drugs are one or more drugs selected from the group comprising anti-IL-6 receptor antibody (e.g. tocilizumab), anti-IL-6 antibody (e.g. siltuximab), anti-TNF antibody (e.g. etanercept, infliximab), anti-IL-1 antibody (e.g. anakinra) and corticosteroids, preferably wherein said corticosteroid is selected from the group comprising cortisone, hydrocortisone, fludrocortisone, dexamethasone, betamethasone, prednisolone, methylprednisolone, and prednisone.

6. Anti-ADM antibody or anti-ADM antibody fragment or anti-ADM non-Ig scaffold for use according to claim 1, wherein said patient is having a level of Pro-Adrenomedullin or fragments thereof in a sample of bodily fluid above a threshold.

7. Anti-ADM antibody or anti-ADM antibody fragment or anti-ADM non-Ig scaffold for use according to claim 6, wherein said Pro-Adrenomedullin or fragment thereof is selected from the group comprising Pro-Adrenomedullin (SEQ ID No.: 2), mature PAMP (SEQ ID No.: 4), PAMP-Gly (SEQ ID No.: 3), MR-proADM (SEQ ID No.: 5), ADM-Gly (SEQ ID No.: 6), mature ADM (SEQ ID No.: 7) and CT-proADM (SEQ ID No.: 8).

8. Anti-ADM antibody or anti-ADM antibody fragment or anti-ADM non-Ig scaffold for use according to claim 7, wherein said fragment of Pro-Adrenomedullin is MR-proADM and the threshold of the level of MR-proADM in a sample of bodily fluid of said patient is between 0.5 and 2 nmol/L, more particular between 0.6 and 1.5 nmol/L, even more particular between 0.7 and 1 nmol/L, most particular said threshold is 0.8 nmol/L
or is an x-fold of the median level of MR-proADM in a healthy population, in particular in the range between 1.2-fold and 4.9-fold, more particular between 1.5-fold and 3.7-fold, even more particular between 1.7-fold and 2.4-fold, most particular said threshold is 2.0-fold of the median of the level of MR-proADM in a healthy population, or wherein said fragment of Pro-Adrenomedullin is mature PAMP and the threshold of the level of mature PAMP in a sample of bodily fluid of said patient is between 0.7 and 1.2 pmol/L, more particular between 0.8 and 1.0 pmol/L, most particular said threshold is 0.9 pmol/L,
or is an x-fold of the mean level of mature PAMP in a healthy population, in particular in the range between the 1.4-fold and 2.4-fold, more particular in the range between 1.6-fold and 2.0-fold, most particular said threshold is the 1.8-fold of the mean of the level of mature PAMP in a healthy population, or wherein said fragment of Pro-Adrenomedullin is PAMP-Gly and the threshold of the level of PAMP-Gly in a sample of bodily fluid of said patient is between 1.5 and 2.4 pmol/L, even more preferred between 1.8 and 2.2 pmol/L, most preferred said threshold is 2.0 pmol/L,
or is an x-fold of the mean level of PAMP-Gly in a healthy population, in particular in the range between the 1.3-fold and 2.1-fold, more particular in the range between 1.6-fold and 1.9-fold, most particular said threshold is the 1.7-fold of the mean of the level of PAMP-Gly in a healthy population, or wherein said fragment of Pro-Adrenomedullin is ADM-Gly and the threshold of the level of ADM-Gly in a sample of bodily fluid of said patient is between 25 and 125 μg/ml, more preferred between and 100 μg/ml, even more particular between 35 and 75 μg/ml, most particular said threshold is 40 μg/ml,
or is an x-fold of the median level of ADM-Gly in a healthy population, in particular in the range between the 0.9-fold and 4.6-fold, more particular in the range between the 1.1-fold and 3.7-fold, more particular in the range between 1.3-fold and 2.8-fold, most particular said threshold is the 1.5-fold of the median of the level of ADM-Gly in a healthy population, or wherein said fragment of Pro-Adrenomedullin is mature ADM and the threshold of the level of mature ADM in a sample of bodily fluid of said patient is between 35 and 125 μg/ml, more particular between 40 and 100 μg/ml, even more particular between 50 and 90 μg/ml, most particular said threshold is 70 μg/ml,
or is an x-fold of the median level of mature ADM in a healthy population, in particular in the range between the 2.9-fold and 7.3-fold, more particular in the range between 3.6-fold and 6.6-fold, most particular said threshold is the 5.1-fold of the median of the level of mature ADM in a healthy population, or wherein said fragment of Pro-Adrenomedullin is CT-proADM and the threshold of the level of CT-proADM in a sample of bodily fluid of said patient is between 75 and 350 pmol/L, more particular between 100 and 250 pmol/L, even more particular between 125 and 200 pmol/L, most particular said threshold is 150 pmol/L,
or is an x-fold of the median level of CT-proADM in a healthy population, in particular in the range between the 1.0-fold and 4.5-fold, more particular in the range between the 1.3-fold and 3.2-fold, more particular in the range between 1.6-fold and 2.6-fold, most particular said threshold is the 1.9-fold of the median of the level of CT-proADM in a healthy population.

9. Anti-ADM antibody or anti-ADM antibody fragment or anti-ADM non-Ig scaffold for use according to cany of claim 6, wherein said sample is a bodily fluid sample selected from the group comprising whole blood, plasma or serum.

10. Anti-ADM antibody or anti-ADM antibody fragment or anti-ADM non-Ig scaffold for according to claim 1, wherein said anti-ADM antibody or anti-ADM fragment or anti-ADM non-Ig scaffold binds to the N-terminal part (amino acids 1-21) of ADM: YRQSMNNFQGLRSFGCRFGTC (SEQ ID No.: 9).

11. Anti-ADM antibody or anti-ADM antibody fragment or anti-ADM non-Ig scaffold for use according to claim 1, wherein said antibody or fragment is a monoclonal antibody or fragment that binds To ADM, wherein the heavy chain comprises the sequences:
CDR1 (SEQ ID No.: 19):
GYTFSRYW
CDR2 (SEQ ID No.: 20):
ILPGSGST
CDR3 (SEQ ID No.: 21):
TEGYEYDGFDY
and wherein the light chain comprises the sequences:
CDR1 (SEQ ID No.: 22):
QSIVYSNGNTY
CDR2:
RVS
CDR3 (SEQ ID No.: 23):
FQGSHIPYT.

12. Anti-ADM antibody or anti-ADM antibody fragment or anti-ADM non-Ig scaffold for use according to claim 1, wherein said anti-ADM antibody or anti-ADM antibody fragment comprises a sequence selected from the group comprising the following sequences as a VH region:
(AM-VH-C)
SEQ ID No.: 24
QVQLQQSGAELMKPGASVKISCKATGYTFSRYWIEWVKQRPGHGLEWIGEILPGSGSTN
YNEKFKGKATITADTSSNTAYMQLSSLTSEDSAVYYCTEGYEYDGFDYWGQGTTLTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPK
(AM-VH1)
SEQ ID No.: 25
QVQLVQSGAEVKKPGSSVKVSCKASGYTFSRYWISWVRQAPGQGLEWMGRILPGSGST
NYAQKFQGRVTITADESTSTAYMELSSLRSEDTAVYYCTEGYEYDGFDYWGQGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK
(AM-VH2-E40)
SEQ ID No.: 26
QVQLVQSGAEVKKPGSSVKVSCKASGYTFSRYWIEWVRQAPGQGLEWMGRILPGSGST
NYAQKFQGRVTITADESTSTAYMELSSLRSEDTAVYYCTEGYEYDGFDYWGQGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK
(AM-VH3-T26-E55)
SEQ ID No.: 27
QVQLVQSGAEVKKPGSSVKVSCKATGYTFSRYWISWVRQAPGQGLEWMGEILPGSGST
NYAQKFQGRVTITADESTSTAYMELSSLRSEDTAVYYCTEGYEYDGFDYWGQGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK
(AM-VH4-T26-E40-E55)
SEQ ID No.: 28
QVQLVQSGAEVKKPGSSVKVSCKATGYTFSRYWIEWVRQAPGQGLEWMGEILPGSGST
NYAQKFQGRVTITADESTSTAYMELSSLRSEDTAVYYCTEGYEYDGFDYWGQGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPK
or a sequence that is >95% identical to it,
and comprises a sequence selected from the group comprising the following sequences as a VL region:
SEQ ID No.: 29 (AM-VL-C):
DVLLSQTPLSLPVSLGDQATISCRSSQSIVYSNGNTYLEWYLQKPGQSPKLLIYRVSNRFS
GVPDRFSGSGSGTDFTLKISRVEAEDLGVYYCFQGSHIPYTFGGGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
(AM-VL1)
SEQ ID No.: 30
DVVMTQSPLSLPVTLGQPASISCRSSQSIVYSNGNTYLNWFQQRPGQSPRRLIYRVSNRDS
GVPDRFSGSGSGTDFTLKISRVEAEDVGVYYCFQGSHIPYTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
(AM-VL2-E40)
SEQ ID No.: 31
DVVMTQSPLSLPVTLGQPASISCRSSQSIVYSNGNTYLEWFQQRPGQSPRRLIYRVSNRDS
GVPDRFSGSGSGTDFTLKISRVEAEDVGVYYCFQGSHIPYTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
or a sequence that is >95% identical to it.

13. Anti-ADM antibody or anti-ADM antibody fragment or anti-ADM non-Ig scaffold for use according to claim 1, wherein said anti-ADM antibody or anti-ADM antibody fragment comprises the following sequence as a heavy chain
SEQ ID No.: 32
QVQLVQSGAEVKKPGSSVKVSCKASGYTFSRYWIEWVRQAPGQGLEWIGEILPGSGSTN
YNQKFQGRVTITADTSTSTAYMELSSLRSEDTAVYYCTEGYEYDGFDYWGQGTTVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKS
RWQQGNVFScSVMHEALHNHYTQKSLSLSPGK
or a sequence that is >95% identical to it,
and comprises the following sequence as a light chain
SEQ ID No.: 33
DVVLTQSPLSLPVTLGQPASISCRSSQSIVYSNGNTYLEWYLQRPGQSPRLLIYRVSNRFS
GVPDRFSGSGSGTDFTLKISRVEAEDVGVYYCFQGSHIPYTFGGGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
or a sequence that is >95% identical to it.


SEQUENCE LISTING (37 sequences)

SEQ ID NO: 1 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 2 - protein, 164 aa, Homo sapiens
ARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 3 - protein, 21 aa, Homo sapiens
ARLDVASEFRKKWNKWALSRG

SEQ ID NO: 4 - protein, 20 aa, Homo sapiens
  MOD_RES 20: note = C-terminal amidation
ARLDVASEFRKKWNKWALSR

SEQ ID NO: 5 - protein, 48 aa, Homo sapiens
ELRMSSSYPTGLADVKAGPAQTLIRPQDMKGASRSPEDSSPDAARIRV

SEQ ID NO: 6 - protein, 53 aa, Homo sapiens
YRQSMNNFQGLRSFGCRFGTCTVQKLAHQIYQFTDKDKDNVAPRSKISPQGYG

SEQ ID NO: 7 - protein, 52 aa, Homo sapiens
  MOD_RES 52: note = C-terminal amidation
YRQSMNNFQGLRSFGCRFGTCTVQKLAHQIYQFTDKDKDNVAPRSKISPQGY

SEQ ID NO: 8 - protein, 38 aa, Homo sapiens
RRRRRSLPEAGPGRTLVSSKPQAHGAPAPPSGSAPHFL

SEQ ID NO: 9 - protein, 21 aa, Homo sapiens
YRQSMNNFQGLRSFGCRFGTC

SEQ ID NO: 10 - protein, 12 aa, Homo sapiens
CTVQKLAHQIYQ

SEQ ID NO: 11 - protein, 12 aa, Homo sapiens
  MOD_RES 12: note = C-terminal amidation
CAPRSKISPQGY

SEQ ID NO: 12 - protein, 19 aa, Homo sapiens
YRQSMNQGSRSNGCRFGTC

SEQ ID NO: 13 - protein, 12 aa, Homo sapiens
CTFQKLAHQIYQ

SEQ ID NO: 14 - protein, 12 aa, Homo sapiens
  MOD_RES 12: note = C-terminal amidation
CAPRNKISPQGY

SEQ ID NO: 15 - protein, 10 aa, Homo sapiens
  MOD_RES 10: note = C-terminal amidation
PRSKISPQGY

SEQ ID NO: 16 - protein, 14 aa, Homo sapiens
YRQSMNNFQGLRSF

SEQ ID NO: 17 - protein, 10 aa, Homo sapiens
YRQSMNNFQG

SEQ ID NO: 18 - protein, 6 aa, Homo sapiens
YRQSMN

SEQ ID NO: 19 - protein, 8 aa, Synthetic construct
GYTFSRYW

SEQ ID NO: 20 - protein, 8 aa, Synthetic construct
ILPGSGST

SEQ ID NO: 21 - protein, 11 aa, Synthetic construct
TEGYEYDGFDY

SEQ ID NO: 22 - protein, 11 aa, Synthetic construct
QSIVYSNGNTY

SEQ ID NO: 23 - protein, 9 aa, Synthetic construct
FQGSHIPYT

SEQ ID NO: 24 - protein, 219 aa, Synthetic construct
QVQLQQSGAELMKPGASVKISCKATGYTFSRYWIEWVKQRPGHGLEWIGEILPGSGSTNYNEKFKGKATITADTSSNTAYMQLSSLTSEDSAVYYCTEGYEYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 25 - protein, 219 aa, Synthetic construct
QVQLVQSGAEVKKPGSSVKVSCKASGYTFSRYWISWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 26 - protein, 219 aa, Synthetic construct
QVQLVQSGAEVKKPGSSVKVSCKASGYTFSRYWIEWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 27 - protein, 219 aa, Synthetic construct
QVQLVQSGAEVKKPGSSVKVSCKATGYTFSRYWISWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 28 - protein, 219 aa, Synthetic construct
QVQLVQSGAEVKKPGSSVKVSCKATGYTFSRYWIEWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 29 - protein, 219 aa, Synthetic construct
DVLLSQTPLSLPVSLGDQATISCRSSQSIVYSNGNTYLEWYLQKPGQSPKLLIYRVSNRFSGVPDRFSGSGSGTDFTLKISRVEAEDLGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 219 aa, Synthetic construct
DVVMTQSPLSLPVTLGQPASISCRSSQSIVYSNGNTYLNWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 219 aa, Synthetic construct
DVVMTQSPLSLPVTLGQPASISCRSSQSIVYSNGNTYLEWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 448 aa, Synthetic construct
QVQLVQSGAEVKKPGSSVKVSCKASGYTFSRYWIEWVRQAPGQGLEWIGEILPGSGSTNYNQKFQGRVTITADTSTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 219 aa, Synthetic construct
DVVLTQSPLSLPVTLGQPASISCRSSQSIVYSNGNTYLEWYLQRPGQSPRLLIYRVSNRFSGVPDRFSGSGSGTDFTLKISRVEAEDVGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 13 aa, Homo sapiens
CAPRSKISPQGYG

SEQ ID NO: 35 - protein, 225 aa, Synthetic construct
QVQLVQSGAEVKKPGSSVKVSCKASGYTFSRYWIEWVRQAPGQGLEWIGEILPGSGSTNYNQKFQGRVTITADTSTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 36 - protein, 118 aa, Homo sapiens
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARYYYYYGMDVWGQGTTVTVSS

SEQ ID NO: 37 - protein, 118 aa, Homo sapiens
QVQLQQSGAELMKPGASVKISCKATGYTFSRYWIEWVKQRPGHGLEWIGEILPGSGSTNYNEKFKGKATITADTSSNTAYMQLSSLTSEDSAVYYCTEGYEYDGFDYWGQGTTLTVSS